(19) United States
(12) Patent Application Publication
(10) Pub. No.: US 2024/0401154 A1
(43) Pub. Date: Dec. 5, 2024

(54) SYNTHESIS AND PREPARATION OF ULTRA-LOW VISCOSITY AND HIGH MAGNETIC SUSCEPTIBILITY MAGNETIC IONIC LIQUIDS

(71) Applicant: Iowa State University Research Foundation, Inc., Ames, IA (US)

(72) Inventors: Jared L. Anderson, Ames, IA (US); Nabeel Mujtaba Abbasi, Ames, IA (US); Victoria Zeger, Ames, IA (US); Shashini De Silva, Ames, IA (US)

(21) Appl. No.: 18/680,822

(22) Filed: May 31, 2024

Related U.S. Application Data
(60) Provisional application No. 63/505,464, filed on Jun. 1, 2023.

(51) Int. Cl. C12Q 1/689; C07F 13/00; C07F 15/04; C07F 15/06

(52) U.S. Cl. CPC C12Q 1/689; C07F 13/005; C07F 15/045; C07F 15/06; C12Q 2521/107; C12Q 2521/507; C12Q 2565/125

(57) ABSTRACT

This disclosure relates to a method and kit for capturing, concentrating, and detecting microbes in a sample using magnetic ionic liquids (MILs) and recombinase polymerase amplification (RPA). Specifically, a method combining magnetic ionic liquid (MIL)-based sample preparation and Recombinase Polymerase Amplification (RPA) for rapid detection of viable microbes in a sample is disclosed. The MILs employed comprise a DGE as the cationic ligand along with an anionic ligand.

### CROSS-REFERENCE TO RELATED APPLICATION

This application claims priority under 35 U.S.C. § 119 to Provisional Application U.S. Ser. No. 63/505,464, filed on Jun. 1, 2023, which is herein incorporated by reference in its entirety including without limitation, the specification, claims, and abstract, as well as any figures, tables, or examples thereof.

### GRANT REFERENCE

This invention was made with government support under Grant number CHE2203891 awarded by the National Science Foundation. The government has certain rights in the invention.

### TECHNICAL FIELD

The present disclosure relates to a method and kit for capturing, concentrating, and detecting microbes in a sample using magnetic ionic liquids (MILs) and recombinase polymerase amplification (RPA). Specifically, a method combining magnetic ionic liquid (MIL)-based sample preparation and Recombinase Polymerase Amplification (RPA) for rapid detection of viable microbes in a sample is disclosed. The MILs employed comprise a DGE as the cationic ligand along with an anionic ligand.

### BACKGROUND

Microbes are everywhere. They can be in drinking water, beverage, river, lake, surface water, soil, air (as in aerosol), food, or food products. They can also be found in biologic fluids or solids from mammals or humans. Most of time, the existence or concentration of microbes does not need to be determined, at least in a short time frame. However, sometime, determination of particular microbes' existence and/or concentration is highly desirable, preferably as soon as possible, for diagnosis, safety, environmental monitoring, product quality control, or manufacturing purposes.

Therefore, rapid, streamlined, and field-deployable methods for detection of some microbes, especially some like Salmonella spp. and other foodborne pathogens are crucial for maintaining public health, identifying pathogens capable of causing illness, ensuring the safety and quality of the food stream, and reducing costs and burdens resulted from pathogen contamination and remedies thereof. Even in some other situations where microbes are not harmful to human, it is still desirable to determine their existence and concentrations for other useful purposes.

(MILs) in separation science to advance the chemical measurement field. MILs are a class of non-molecular solvents that possess low melting points, tunable viscosities, and can be readily manipulated using an applied magnetic field. While MILs have been studied and utilized for extracting microbes, there are a number of ways that MILs can continue to be improved. Two areas where MILs can continue to be improved are physicochemical properties and higher magnetic susceptibility.

Owing to advancements made in the design of their chemical structures, different classes of MILs have evolved over the years, with key features that can influence specific MIL properties having been identified. For example, bulky alkyl functional groups in the cation/anion can enhance thermal stability and hydrophobicity of MILs, while employing anionic ligands containing trifluoromethyl moieties (CF₃) can expand their hydrophobic nature and result in reduced MIL viscosity. Aromatic substituents in ligands have also been found to enhance the thermal stability of MILs, but generally yield highly viscous solvents. While their magnetic properties are primarily controlled by the type and number of paramagnetic centers, their viscosity and solvation properties (e.g., dipolar and dispersive-type interactions) can also be influenced by the metal employed.

The first reported MIL, 1-butyl-3-methylimidazolium tetrachloroferrate (III) ([C₄mim⁺][FeCl₄⁻]), was shown to have a viscosity of only 18 cP, but is plagued by stability issues in aqueous environments. Given hydrolysis of the [FeCl₄⁻] anion in water, the [MnCl₄²⁻], [CoCl₄²⁻], and [GdCl₆³⁻] anions have demonstrated improved stability, but also produce MILs with viscosities as high as 123,500 cP between 273 and 373 K.

To modulate MIL viscosity, several approaches have been used to date. MILs mixed with water and organic solvents have been employed to facilitate the lowering of their charge density and viscosity. However, this strategy can only be applied to hydrophilic MILs. Additionally, the unique physico-chemical properties of MILs are lost when they are diluted with traditional solvents beyond a specific concentration range. Generally, anions comprised of hexafluoroacetylacetonate ligands [hfacac⁻] and bis[(trifluoromethyl)sulfonyl]imide [NTf₂⁻] are employed to produce low viscosity MILs. However, event these MILs do not have the ultra-low viscosity needed for certain applications and have also not had as high of magnetic susceptibility when paired with magnetic metals. Thus, there remains a need for improved MILs.

An object and/or advantage of this disclosure is providing MILs with ultra-low viscosities.

A further object and/or advantage of this disclosure is providing MILs with high magnetic susceptibility.

A further object and/or advantage of this disclosure is providing MILs which are suitable with a variety of solvents.

### BRIEF DESCRIPTION OF THE FIGURES

The patent or application file contains at least one drawing executed in color. Copies of this patent or patent application publication with color drawing(s) will be provided by the Office upon request and payment of the necessary fee.

The following drawings form part of the specification and are included to further demonstrate certain embodiments. In some instances, embodiments can be best understood by referring to the accompanying figures in combination with the detailed description presented herein. The description and accompanying figures may highlight a certain specific example, or a certain embodiment. However, one skilled in the art will understand that portions of the example or embodiment may be used in combination with other examples or embodiments.

FIG. 1 shows the structures of the four DGE ligands subject to this disclosure and assessed during the examples provided herein.

FIG. 2A shows the chemical structure of MILs containing transition metals in the cation and anion.

FIG. 2B shows the chemical structure of MILs containing transition and rare-earth metals in the cation and anion.

FIG. 3 shows homogenous and mixed metal MIL combinations, with reference to panels provided in FIG. 2A and FIG. 2B.

FIG. 4 shows the ¹H NMR spectrum of the dihexyl-2,2′-oxidiacetate (C₆-DGE) ligand.

FIG. 5 shows the ¹³C NMR spectrum of the dihexyl-2,2′-oxidiacetate (C₆-DGE) ligand.

FIG. 6 shows the ¹H NMR spectrum of the dioctyl-2,2′-oxidiacetate (C₈-DGE) ligand.

FIG. 7 shows the ¹³C NMR spectrum of the dioctyl-2,2′-oxidiacetate (C₈-DGE) ligand.

FIG. 8 shows the ¹H NMR spectrum of the dicyclohexyl-2,2′-oxidiacetate (CycloC₆-DGE) ligand.

FIG. 9 shows the ¹³C NMR spectrum of the dicyclohexyl-2,2′-oxidiacetate (CycloC₆-DGE) ligand.

FIG. 10 shows the ¹H NMR spectrum of the dioctylfuran-2,5-dicarboxylate (FuranC₈-DGE) ligand.

FIG. 11 shows the ¹³C NMR spectrum of the dioctylfuran-2,5-dicarboxylate (FuranC₈-DGE) ligand.

FIGS. 12A and 12B show the magnetization of Ni-based (FIG. 12A) and Mn-based (FIG. 12B) MILs plotted as a function of temperature.

FIG. 13 shows the volatilization/degradation of the [Co(C₆-DGE)₆][Co(hfacac)₃]₂ MIL as a function of temperature measured using TGA.

FIG. 14 shows the volatilization/degradation of the [Ni(C₆-DGE)₆][Ni(hfacac)₃]₂ MIL as a function of temperature measured using TGA.

FIG. 15 shows the volatilization/degradation of the [Mn(C₆-DGE)₆][Mn(hfacac)₃]₂ MIL as a function of temperature measured using TGA.

FIG. 16 shows the volatilization/degradation of the [Co(C₈-DGE)₆][Co(hfacac)₃]₂ MIL as a function of temperature measured using TGA.

FIG. 17 shows the volatilization/degradation of the [Ni(C₈-DGE)₆][Ni(hfacac)₃]₂ MIL as a function of temperature measured using TGA.

FIG. 18 shows the volatilization/degradation of the [Mn(C₈-DGE)₆][Mn(hfacac)₃]₂ MIL as a function of temperature measured using TGA.

FIG. 19 shows the volatilization/degradation of the [Co(CycloC₆-DGE)₆][Co(hfacac)₃]₂ MIL as a function of temperature measured using TGA.

FIG. 20 shows the volatilization/degradation of the [Ni(CycloC₆-DGE)₆][Ni(hfacac)₃]₂ MIL as a function of temperature measured using TGA.

FIG. 21 shows the volatilization/degradation of the [Mn(CycloC₆-DGE)₆][Mn(hfacac)₃]₂ MIL as a function of temperature measured using TGA.

FIG. 22 shows the volatilization/degradation of the [Co(FuranC₈-DGE)₆][Co(hfacac)₃]₂ MIL as a function of temperature measured using TGA.

FIG. 23 shows the volatilization/degradation of the [Ni(FuranC₈-DGE)₆][Ni(hfacac)₃]₂ MIL as a function of temperature measured using TGA.

FIG. 24 shows the volatilization/degradation of the [Mn(FuranC₈-DGE)₆][Mn(hfacac)₃]₂ MIL as a function of temperature measured using TGA.

FIG. 25 shows the volatilization/degradation of the [Co(C₈-DGE)₆][Dy(hfacac)₄]₂ MIL as a function of temperature measured using TGA.

FIG. 26 shows the volatilization/degradation of the [Co(C₈-DGE)₆][Gd(hfacac)₄]₂ MIL as a function of temperature measured using TGA.

FIG. 27 shows the volatilization/degradation of the [Co(C₈-DGE)₆][Ho(hfacac)₄]₂ MIL as a function of temperature measured using TGA.

FIGS. 28A and 28B show the FID detector response as a function of temperature for select MILs.

The application also includes tables in the body of the application. Summaries of those tables are included below. The tables are non-limiting and used to illustrate preferred embodiments disclosed herein.

TABLE 1 shows the synthesized MILs with their respective viscosities, theoretical effective paramagnetic moments, and effective magnetic moments.

TABLE 2 shows the individual viscosities of previously studied DGA ligands.

TABLE 3 shows the solubility of each MIL as well as the temperature of onset MIL volatilization. Additionally, TABLE 3 provides the onset temperature of MIL volatilization/degradation obtained from TGA measurements at 10% weight loss.

### DETAILED DESCRIPTION

The present disclosure relates to a method for extracting, concentrating, detecting viable microbes from a sample. The embodiments of the methods and kits are not limited to any particular metal ion, chelating species, microbe, extracting medium, RPA method, or detection methods for amplicons which can vary and are understood by skilled artisans based on the present disclosure. Beneficially, the MILs and related methods disclosed herein can include higher magnetic susceptibility as well as greater compatibility with bioanalytical assays and chromatographic separations.

So that the present disclosure may be more readily understood, certain terms are first defined. Unless defined otherwise, all technical and scientific terms used herein have the same meaning as commonly understood by one of ordinary skill in the art to which embodiments of the disclosed MILs and methods pertain. Many methods and materials similar, modified, or equivalent to those described herein can be used in the practice of the embodiments of the disclosed MILs and methods without undue experimentation. The preferred materials and methods are described herein. In describing and claiming the embodiments of the disclosed MILs and methods, the following terminology will be used in accordance with the definitions set out below.

### Definitions

It is to be understood that all terminology used herein is for the purpose of describing particular embodiments only and is not intended to be limiting in any manner or scope. For example, as used in this specification and the appended claims, the singular forms “a,” “an” and “the” can include plural referents unless the content clearly indicates otherwise. Thus, for example, reference to “a cell” includes a single cell, as well as two or more cells. Similarly, the word “or” is intended to include “and” unless the context clearly indicate otherwise. The word “or” means any one member of a particular list and also includes any combination of members of that list. Further, all units, prefixes, and symbols may be denoted in its SI accepted form.

Numeric ranges recited within the specification are inclusive of the numbers defining the range and include each integer within the defined range. Throughout this disclosure, various embodiments of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 1½, and 4¾. This applies regardless of the breadth of the range.

The term “about,” as used herein, refers to variations in size, distance or any other types of measurements that can be resulted from the inherent heterogeneous nature of the measured objects and imprecise nature of the measurements themselves. The term “about” also encompasses variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods, and the like. The term “about” also encompasses amounts that differ due to different equilibrium conditions for a composition resulting from a particular initial mixture. Whether or not modified by the term “about”, the claims include equivalents to the quantities.

As used herein the term, “diagnostically useful cellular components” includes, but is not limited to, DNA, RNA, other nucleic acids, proteins, enzymes, lipids or cell wall materials characteristic of the target organism.

The term “independently” means that where more than one substituent is selected from a number of possible substituents, those substituents may be the same or different.

As used herein, “substituted” refers to an organic group as defined below (i.e., an alkyl group) in which one or more bonds to a hydrogen atom contained therein are replaced by a bond to non-hydrogen or non-carbon atoms. Substituted groups also include groups in which one or more bonds to carbon(s) or hydrogen(s) atom replaced by one or more bonds, including double or triple bonds, to a heteroatom. Thus, a substituted group is substituted with one or more substituents, unless otherwise specified. A substituted group can be substituted with 1, 2, 3, 4, 5, or 6 substituents.

Substituted ring groups include rings and ring systems in which a bond to a hydrogen atom is replaced with a bond to a carbon atom. Therefore, substituted cycloalkyl, aryl, heterocyclyl, and heteroaryl groups may also be substituted with substituted or unsubstituted alkyl, alkenyl, and alkynyl groups are as defined herein.

As used herein, the term “alkyl” or “alkyl groups” refers to saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl, cyclopentyl, cyclohexyl, cycloheptyl, cyclooctyl, etc.), branched-chain alkyl groups (e.g., isopropyl, tert-butyl, sec-butyl, isobutyl, etc.), and alkyl-substituted alkyl groups (e.g., alkyl-substituted cycloalkyl groups and cycloalkyl-substituted alkyl groups).

Unless otherwise specified, the term “alkyl” includes both “unsubstituted alkyls” and “substituted alkyls.” As used herein, the term “substituted alkyls” refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), imino, sulfhydryl, alkylthio, arylthio, thiocarboxylate, sulfates, alkylsulfinyl, sulfonates, sulfamoyl, sulfonamido, nitro, trifluoromethyl, cyano, azido, heterocyclic, alkylaryl, or aromatic (including heteroaromatic) groups.

In some embodiments, substituted alkyls can include a heterocyclic group. As used herein, the term “heterocyclic group” includes closed ring structures analogous to carbocyclic groups in which one or more of the carbon atoms in the ring is an element other than carbon, for example, nitrogen, sulfur or oxygen. Heterocyclic groups may be saturated or unsaturated. Exemplary heterocyclic groups include, but are not limited to, aziridine, ethylene oxide (epoxides, oxiranes), thiirane (episulfides), dioxirane, azetidine, oxetane, thietane, dioxetane, dithietane, dithiete, azolidine, pyrrolidine, pyrroline, oxolane, dihydrofuran, and furan.

Alkenyl groups or alkenes are straight chain, branched, or cyclic alkyl groups having 2 to about 30 carbon atoms, and further including at least one double bond. In some embodiments, alkenyl groups have from 2 to about 20 carbon, or typically, from 2 to 10 carbon atoms. Alkenyl groups may be substituted or unsubstituted. Alkenyl groups may be substituted similarly to alkyl groups.

As used herein, the terms “alkylene”, cycloalkylene “, alkynylene, and alkenylene”, alone or as part of another substituent, refer to a divalent radical derived from an alkyl, cycloalkyl, or alkenyl group, respectively, as exemplified by —CH₂CH₂CH₂—. For alkylene, cycloalkylene, alkynylene, and alkenylene groups, no orientation of the linking group is implied.

As used herein, “aryl” or “aromatic” groups are cyclic aromatic hydrocarbons that do not contain heteroatoms. Aryl groups include monocyclic, bicyclic, and polycyclic ring systems. Thus, aryl groups include, but are not limited to, phenyl, azulenyl, heptalenyl, biphenylenyl, indacenyl, florenyl, phenanthrenyl, triphenylenyl, pyrenyl, naphthacenyl, chrysenyl, biphenyl, anthracenyl, indenyl, indanyl, pentalenyl, and naphthyl groups. In some embodiments, aryl groups contain 6-14 carbons, in others from 6 to 12 or 6-10 carbon atoms in the ring portions of the groups. The phrase “aryl groups” includes groups containing fused rings, such as fused aromatic-aliphatic ring systems. Aryl groups may be substituted or unsubstituted.

As used herein, “hfacac” and “hfacac⁻” refer to hexafluoroacetylacetonate and its anion, which can be used herein an anionic ligand.

As used herein, “extract” means that the microbes or their diagnostically useful cellular components, including, but not limited to DNA, RNA, other nucleic acids, proteins, enzymes, lipids or cell wall materials characteristic of the target organism, in the sample migrates onto or into the MIL during the contact time and then from the MIL to the extracting medium during the extracting step. After the “contacting” step and before the “extracting” step, the MIL can be mixed well with the sample, if the sample is liquid or contains liquid. In some situations, if the sample cannot mix with MIL liquid thoroughly, water or a solvent can be added to the sample or the mixture of the sample and MIL, so the added water and solvent can be the medium for the microbes to migrate from the sample to MIL.

### DETAILED DISCLOSURE

Cationic ligands incorporating both aromatic and aliphatic alkyl substituents were employed to prepare MILs that simultaneously contain 2 different types of metal centers in their chemical structure, resulting in effective magnetic moments (μ_{eff}) as high as 15.56 Bohr magnetons (μ_{B}). Varying the length of alkyl moieties or the incorporation of cyclic functional groups in the cationic ligands was found to influence MIL thermal stability, with those comprised of octyl substituents being stable at temperatures up to 177° C. The new generation of MILs excel at offering ideal physico-chemical properties often sought out in designer solvents. This study explores the distinct design of DGE-based MILs and demonstrates that such combinations produce compounds that are less viscous compared to many organic solvents, making them highly appealing in catalysis, organic synthesis, as well as chemical and electrochemical separations as a sustainable replacement for traditional solvents, where their paramagnetic properties can be readily exploited.

As disclosed herein, diglycolic acid esters can be chelated to transition metals to form bulky cationic ligands, which are preferably paired with an anionic ligand (most preferably hfacac⁻ metal chelates) to produce MILs. Beneficially, these MILs comprising DGE have low viscosities. Further, embedding paramagnetic centers in both the cation/anion results in enhanced magnetic susceptibilities (μ_{eff} of up to 15.56μ_{B}) and incorporates characteristics of multiple metal centers in mixed metal MILs. The MILs disclosed herein can provide strong hydrophobicity and can be soluble in non-polar solvents (hexane, benzene, etc.) at concentrations of up to 50% (w/v) MIL-to-solvent ratio and insoluble in water at 0.01% (w/v).

In one aspect, disclosed herein is a method of extracting, concentrating, detecting viable microbes from a sample, the method comprises contacting a sample with a magnetic ionic liquid (MIL) for the period of a contacting time; extracting the microbes or their diagnostically useful cellular components, including, but not limited to DNA, RNA, or other nucleic acids, from the magnetic ionic liquid using an aqueous extracting medium to generate an extracted sample; and using Recombinase Polymerase Amplification (RPA) on the extracted sample for amplifying DNA or RNA of the microbes, wherein the sample comprises viable microbes.

In another aspect, disclosed herein is a method of extracting, concentrating, detecting viable microbes from a sample, the method comprises contacting a sample with a magnetic ionic liquid (MIL) for the period of a contacting time; extracting the microbes or their diagnostically useful cellular components, including, but not limited to DNA, RNA, or other nucleic acids, from the magnetic ionic liquid using an aqueous extracting medium to generate an extracted sample; and using Recombinase Polymerase Amplification (RPA) on the extracted sample for amplifying DNA or RNA of the microbes; wherein the RPA is carried out with a power-free heat source, wherein the sample comprises viable microbes.

In yet another aspect, disclosed herein is a method of extracting, concentrating, detecting viable microbes from a sample, the method comprises contacting a sample with a magnetic ionic liquid (MIL) for the period of a contacting time; extracting the microbes or their diagnostically useful cellular components, including, but not limited to DNA, RNA, or other nucleic acids, from the magnetic ionic liquid using an aqueous extracting medium to generate an extracted sample; wherein the extracting medium is a Luria-Bertani-derived nutrient broth comprising more than 10 g/L of tryptone, 10 g/L of NaCl, 5 g/L of yeast extract, or combination thereof; and using Recombinase Polymerase Amplification (RPA) on the extracted sample for amplifying DNA or RNA of the microbes, wherein the sample comprises viable microbes.

In yet another aspect, disclosed herein is a method of extracting, concentrating, detecting viable microbes from a sample, the method comprises contacting a sample with a magnetic ionic liquid (MIL) for the period of a contacting time; extracting the microbes or their diagnostically useful cellular components, including, but not limited to DNA, RNA, or other nucleic acids, from the magnetic ionic liquid using an aqueous extracting medium to generate an extracted sample; and using Recombinase Polymerase Amplification (RPA) on the extracted sample for amplifying DNA or RNA of the microbes, wherein the sample comprises viable microbes: wherein the microbes are Salmonella, and wherein RPA is carried out with a primer for Salmonella-specific DLH gene.

### Magnetic Ionic Liquids (MILs)

Reduction and customization of MIL viscosity is often of significant interest in many applications. For MILs to function as solvents, extractants or reaction media, low viscosities are often preferred as it facilitates ease of pipetting²⁵ and allows rapid transport/exchange of materials. In separation systems involving two immiscible solvents, low viscosity enables ease of MIL dispersion and subsequent phase separation without the need of tedious centrifugation and ultrasonication steps. In addition, measuring very small quantities of MILs (e.g., microliter volumes) can be challenging due to their high viscosity, which can adversely impact accuracy, precision, and method reproducibility in applications that require high volume precision. However, MILs generally resemble oils and possess viscosities that are two to three orders of magnitude higher than that of organic solvents.

As noted above, anions comprised of hexafluoroacetylacetonate ligands [hfacac] and bis[(trifluoromethyl)sulfonyl]imide [NTf₂⁻] have been employed to produce low viscosity MILs. A related disclosure which discloses earlier MILs and ligands forming MILs is found in US Pat. Publ. No. 2020/0325525A1, the detailed disclosure and figures are herein incorporated by reference in their entirety. With such developments, a new class of MILs comprised of [hfacac⁻] metal chelates was recently reported that simultaneously incorporate multiple lanthanide and transition metal centers in both the cation and anion. Given that [hfacac⁻] metal chelates employed as anions can yield lower viscosities, the cationic component of these MILs remains a key target for further chemical structure modification. To date, only diglycolamides (DGAs) have been used as cationic ligands in these MILs and no other multi-dentate chelating species have been explored to lower their viscosity.

We have identified that diglycolic acid esters (DGEs) can be used as cationic ligands and provide ultra-low viscosity as well as other physiochemical properties.

The MILs comprise a cationic ligand having a paramagnetic center and an anionic ligand having a paramagnetic center. According to this disclosure, preferred ligands include a paramagnetic cationic DGE ligands and a paramagnetic anionic hfacac⁻ ligands.

The MILs include both a paramagnetic cationic ligand and a paramagnetic anionic ligand. The metal species retained by the ligands can be the same or different (e.g., a single species is retained in both the cationic ligand and the anionic ligand, or different metal species are retained in the anionic ligand as opposed to the cationic ligand). In some embodiments, mixtures of metals are retained in either the cationic ligand, anionic ligand, or both. Preferred metals are discussed following the discussion of ligands.

Preferred MILs include both a cationic ligand and an anionic ligand, each of which retains a paramagnetic metal. Most preferably the MILs comprise a paramagnetic cationic ligand and a paramagnetic anionic ligand, including, but not limited to the following combinations:

| Homogenous MIL combinations (represented by panel A) | | Mixed metal MIL combinations (represented by panel B) |
|---|---|---|
| 1. [Co( [symbol] ) [symbol] ][Co(hfacac) [symbol] ] [symbol] | 7. [Co(Cyclo [symbol] ) [symbol] ][Co(hfacac) [symbol] ] [symbol] | 13. [Co( [symbol] ) [symbol] ][ [symbol] (hfacac) [symbol] ] [symbol] |
| 2. [Ni( [symbol] ) [symbol] ][Ni(hfacac) [symbol] ] [symbol] | 8. [Ni(Cyclo [symbol] ) [symbol] ][Ni(hfacac) [symbol] ] [symbol] | 14. [Co( [symbol] ) [symbol] ][ [symbol] (hfacac) [symbol] ] [symbol] |
| 3. [Mn( [symbol] ) [symbol] ][Mn(hfacac) [symbol] ] [symbol] | 9. [Mn(Cyclo [symbol] ) [symbol] ][Mn(hfacac) [symbol] ] [symbol] | 15. [Co( [symbol] ) [symbol] ][ [symbol] (hfacac) [symbol] ] [symbol] |
| 4. [Co( [symbol] ) [symbol] ][Co(hfacac) [symbol] ] [symbol] | 10. [Co( [symbol] ) [symbol] ][Co(hfacac) [symbol] ] [symbol] | |
| 5. [Ni( [symbol] ) [symbol] ][Ni(hfacac) [symbol] ] [symbol] | 11. [Ni( [symbol] ) [symbol] ][Ni(hfacac) [symbol] ] [symbol] | |
| 6. [Mn( [symbol] ) [symbol] ][Mn(hfacac) [symbol] ] [symbol] | 12. [Mn( [symbol] ) [symbol] ][Mn(hfacac) [symbol] ] [symbol] | |

[symbol]

indicates data missing or illegible when filed

### Cationic Ligands

Preferred cationic ligands are diglycolic acid esters which have the following structure:

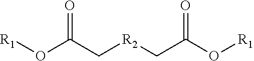

According to the cationic ligand structure, R1 is an organic substituent. Preferred organic substituents include, but are not limited to, substituted organic groups. Preferred organic groups which can be substituted include, but are not limited to, alkyl groups (including linear and branched moieties), ethoxylated chains, alkene chains, carbon chains comprised of alkyl and alkene moieties, and mixtures of thereof. Preferably, organic substituent has from about 2 carbons to about 24 carbons, from about 3 carbons to about 22 carbons, from about 4 carbons to about 20 carbons.

Preferred R¹ organic substituents include, but are not limited to:

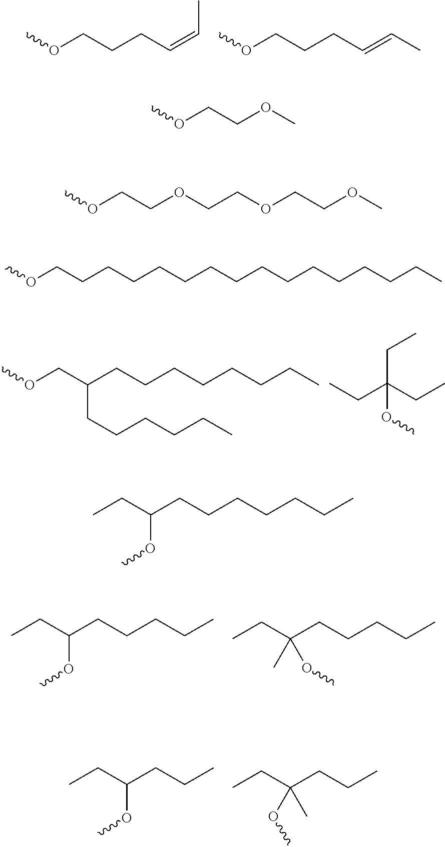

According to the anionic ligand structure, R² is an N—CH₃, O, S, or thiophene. Embodiments of the ligand backbone structure (which can be substituted by removal and replacement of the OH groups and Cl anions shown below with organic substituents (R¹)) are as follows:

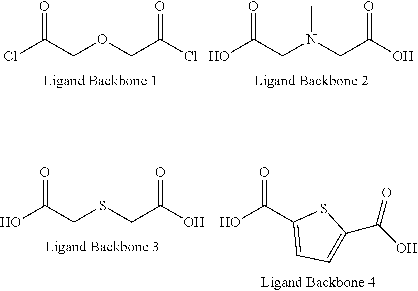

where Ligand Backbone 1 has an oxygen (O) as R², Ligand Backbone 2 has a N—CH3 group as R², Ligand Backbone 3 has a sulfur(S) as R², and Ligand Backbone 4 has a thiophene group as R².

Preferred cationic ligands include, but are not limited to, the following DGE ligands:

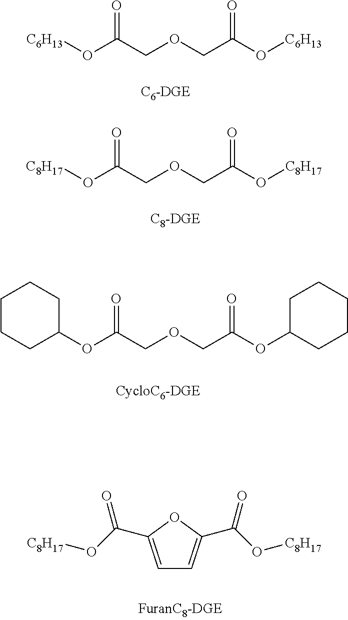

Beneficially, these cationic ligands chelate a metal, preferably a paramagnetic metal. Preferred metals are disclosed below.

### Anionic Ligands

Preferred anionic ligands include hfacac⁻ metal chelates and have the following general formula:

[M(Y)ₓ⁻]
wherein M is paramagnetic transition metal or rare earth metal ion; and Y is a chelating agent having the general formula:

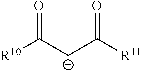

wherein each of R¹⁰ and R¹¹ is independently a substituted or unsubstituted methyl, phenyl, thiophenyl, napthyl, alkyl, or aryl group; and x is 3 or 4.

In a preferred embodiment, the MIL comprises a paramagnetic anionic ligand as shown below:

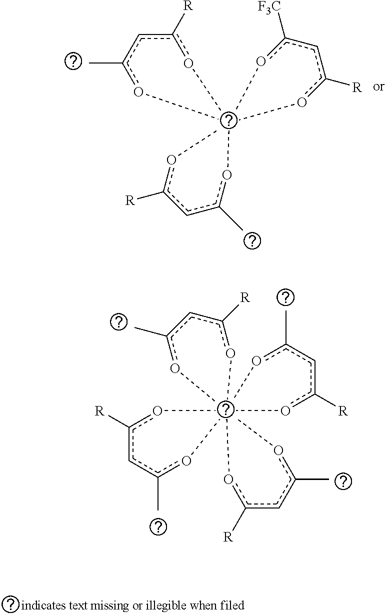

wherein R is one or more of the following:

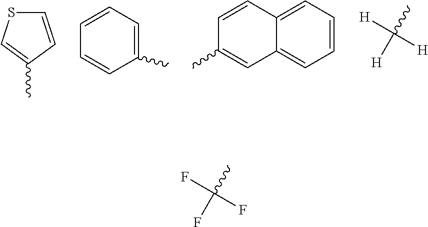

In some embodiments, for the magnetic ionic liquid disclosed herein, R¹⁰ and R¹¹ are independently a methyl, phenyl, thiophenyl, napthyl, alkyl, or aryl group substituted by one or more electron withdrawing halogens or other groups. In some other embodiments, R¹⁰ and R¹¹ are independently a C1-C4 alkyl group substituted by one or more electron withdrawing halogens or other groups. In some other embodiments, R¹⁰ and R¹¹ are independently a CH₃, CHF₂, CH₂F, or CF₃ group. In yet some other embodiments, R¹⁰ and R¹¹ are independently a CF₃ group.

In some other embodiments, for the magnetic ionic liquid disclosed herein, the anionic ligand comprises [Co(hfacac)₃⁻], [Ni(hfacac)₃⁻], ([Mn(hfacac)₃⁻]), ([Dy(hfacac)₄⁻]), ([Gd(hfacac)₄⁻]), ([Nd(hfacac)₄⁻]), or combination thereof, wherein hfacac is

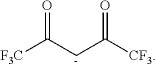

In some embodiments, for the MILs disclosed herein the anionic ligand comprises

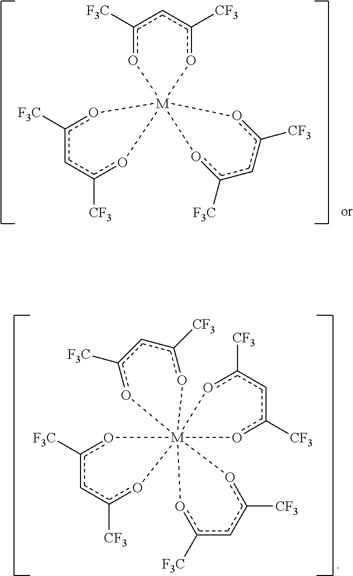

An example synthesis route for a preferred anionic ligand is:

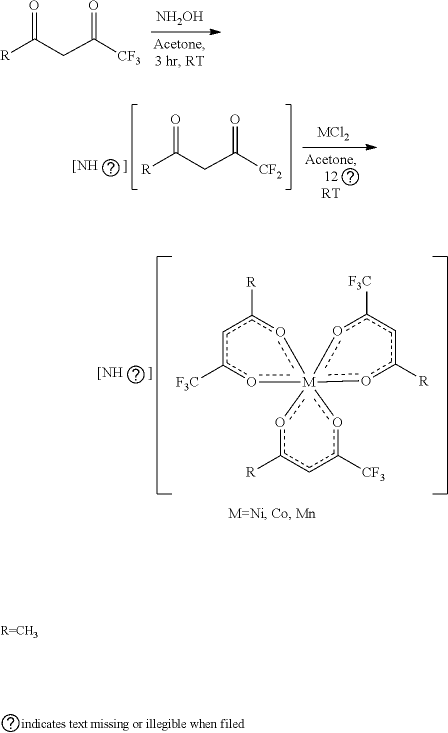

### Metals

Beneficially, the each of the ligands (i.e., the anionic ligand and cationic ligand) comprise one or more metals. Preferred metals include, but are not limited to, nickel (Ni), copper (Cu), cobalt (Co), iron (Fe), manganese (Mn), chromium (Cr), vanadium (V), dysprosium (Dy), holmium (Ho), gadolinium (Gd), neodymium (Nd), europium (Eu), cerium (Ce), thulium (Tm), ruthenium (Re), terbium (Tb), erbium (Er), ytterbium (Yb), and salts or mixtures thereof. Notably, the metal species retained in the anionic ligand can be the same or different than the metal species retained in the cationic ligand.

Metals suitable for the MIL are those having magnetic susceptibility. Those listed in the above paragraph have magnetic susceptibility. Preferably the metal has a magnetic susceptibility of at least about 1000 10⁻⁶ cm³ mol⁻¹, of at least about 1500 10⁻⁶ cm³ mol⁻¹, of at least about 2000 10⁻⁶ cm³ mol⁻¹, of at least about 2500 10⁻⁶ cm³ mol⁻¹, of at least about 3000 10⁻⁶ cm³ mol⁻¹, of at least about 3500 10⁻⁶ cm³ mol⁻¹, of at least about 4000 10⁻⁶ cm³ mol⁻¹, of at least about 4500 10⁻⁶ cm³ mol⁻¹, of at least about 5000 10⁻⁶ cm³ mol⁻¹, of at least about 5500 10⁻⁶ cm³ mol⁻¹, of at least about 6000 10⁻⁶ cm³ mol⁻¹, of at least about 6500 10⁻⁶ cm³ mol⁻¹, of at least about 7000 10⁻⁶ cm³ mol⁻¹, of at least about 7500 10⁻⁶ cm³ mol⁻¹, of at least about 8000 10⁻⁶ cm³ mol⁻¹, of at least about 8500 10⁻⁶ cm³ mol⁻¹, of at least about 9000 10⁻⁶ cm³ mol⁻¹, or of at least about 10,000 10⁻⁶ cm³ mol⁻¹. It should be recognized that the degree magnetic susceptibility may be specific to particular applications and needs. Thus, in certain circumstances a lesser degree of magnetic susceptibility may be suitable whereas in others, a higher degree may be desired. As such, the metal chosen can be selected based on balancing considerations between magnetic susceptibility and cost.

Most preferred metals include, but are not limited to, those disclosed in the following table:

### Magnetic Moment

| Name | Magnetic Susceptibility [symbol] |
|---|---|
| Cobalt (II) Chloride (hexahydrate) | +12660 (+9710) |
| Nickel (II) Chloride (hexahydrate) | +6145 (+4240) |
| Manganese (II) Chloride (hexahydrate) | +14350 (+14600) |
| Copper (II) Chloride | +1080 |
| Vanadium (III) Chloride | +3030 |
| Vanadium (II) Chloride | +2410 |
| Chromium (III) Chloride | +6350 |
| Chromium (II) Chloride | +7230 |
| Ruthenium (III) Chloride | +1998 |
| Gadolinium (III) Chloride | +27930 |
| Cerium (III) Chloride | +2490 |

[symbol]

indicates data missing or illegible when filed

Beneficially, the MILs disclosed herein have ultra low viscosity. In some embodiments, the MILs disclosed herein have a viscosity of less than about 600 cp at the temperature of about 23.6° C., a viscosity of less than about 550 cp at the temperature of about 23.6° C., a viscosity of less than about 500 cp at the temperature of about 23.6° C., a viscosity of less than about 450 cp at the temperature of about 23.6° C., a viscosity of less than about 400 cp at the temperature of about 23.6° C., a viscosity of less than about 350 cp at the temperature of about 23.6° C., a viscosity of less than about 300 cp at the temperature of about 23.6° C., less than about 250 cp at the temperature of about 23.6° C., less than about 200 cp at the temperature of about 23.6° C., less than about 190 cp at the temperature of about 23.6° C., less than about 180 cp at the temperature of about 23.6° C., less than about 170 cp at the temperature of about 23.6° C., less than about 160 cp at the temperature of about 23.6° C., equal to or less than about 159 cp at the temperature of about 23.6° C., equal to or less than about 158 cp at the temperature of about 23.6° C., equal to or less than about 179 cp at the temperature of about 23.6° C., equal to or less than about 156 cp at the temperature of about 23.6° C., equal to or less than about 155 cp at the temperature of about 23.6° C., equal to or less than about 154 cp at the temperature of about 23.6° C., equal to or less than about 153 cp at the temperature of about 23.6° C., equal to or less than about 152 cp at the temperature of about 23.6° C., equal to or less than about 151 cp at the temperature of about 23.6° C., equal to or less than about 150 cp at the temperature of about 23.6° C., equal to or less than about 149 cp at the temperature of about 23.6° C., equal to or less than about 148 cp at the temperature of about 23.6° C., equal to or less than about 147 cp at the temperature of about 23.6° C., equal to or less than about 146 cp at the temperature of about 23.6° C., equal to or less than about 145 cp at the temperature of about 23.6° C., equal to or less than about 144 cp at the temperature of about 23.6° C., equal to or less than about 143 cp at the temperature of about 23.6° C., equal to or less than about 142 cp at the temperature of about 23.6° C., equal to or less than about 141 cp at the temperature of about 23.6° C., equal to or less than about 140 cp at the temperature of about 23.6° C., equal to or less than about 139 cp at the temperature of about 23.6° C., equal to or less than about 138 cp at the temperature of about 23.6° C., equal to or less than about 137 cp at the temperature of about 23.6° C., equal to or less than about 136 cp at the temperature of about 23.6° C., equal to or less than about 135 cp at the temperature of about 23.6° C., equal to or less than about 134 cp at the temperature of about 23.6° C., equal to or less than about 133 cp at the temperature of about 23.6° C., equal to or less than about 132 cp at the temperature of about 23.6° C., equal to or less than about 131 cp at the temperature of about 23.6° C., equal to or less than about 130 cp at the temperature of about 23.6° C., equal to or less than about 129 cp at the temperature of about 23.6° C., equal to or less than about 128 cp at the temperature of about 23.6° C., equal to or less than about 127 cp at the temperature of about 23.6° C., equal to or less than about 126 cp at the temperature of about 23.6° C., equal to or less than about 125 cp at the temperature of about 23.6° C., equal to or less than about 124 cp at the temperature of about 23.6° C., equal to or less than about 123 cp at the temperature of about 23.6° C., equal to or less than about 122 cp at the temperature of about 23.6° C., equal to or less than about 121 cp at the temperature of about 23.6° C., equal to or less than about 120 cp at the temperature of about 23.6° C., equal to or less than about 119 cp at the temperature of about 23.6° C., equal to or less than about 118 cp at the temperature of about 23.6° C., equal to or less than about 117 cp at the temperature of about 23.6° C., equal to or less than about 116 cp at the temperature of about 23.6° C., equal to or less than about 115 cp at the temperature of about 23.6° C., equal to or less than about 114 cp at the temperature of about 23.6° C., equal to or less than about 113 cp at the temperature of about 23.6° C., equal to or less than about 112 cp at the temperature of about 23.6° C., equal to or less than about 111 cp at the temperature of about 23.6° C., equal to or less than about 110 cp at the temperature of about 23.6° C., equal to or less than about 109 cp at the temperature of about 23.6° C., equal to or less than about 108 cp at the temperature of about 23.6° C., equal to or less than about 107 cp at the temperature of about 23.6° C., equal to or less than about 106 cp at the temperature of about 23.6° C., equal to or less than about 105 cp at the temperature of about 23.6° C., equal to or less than about 104 cp at the temperature of about 23.6° C., equal to or less than about 103 cp at the temperature of about 23.6° C., equal to or less than about 102 cp at the temperature of about 23.6° C., equal to or less than about 101 cp at the temperature of about 23.6° C., equal to or less than about 100 cp at the temperature of about 23.6° C., equal to or less than about 99 cp at the temperature of about 23.6° C., equal to or less than about 98 cp at the temperature of about 23.6° C., equal to or less than about 97 cp at the temperature of about 23.6° C., equal to or less than about 96 cp at the temperature of about 23.6° C., equal to or less than about 95 cp at the temperature of about 23.6° C., equal to or less than about 94 cp at the temperature of about 23.6° C., equal to or less than about 93 cp at the temperature of about 23.6° C., equal to or less than about 92 cp at the temperature of about 23.6° C., equal to or less than about 91 cp at the temperature of about 23.6° C., equal to or less than about 90 cp at the temperature of about 23.6° C., equal to or less than about 89 cp at the temperature of about 23.6° C., equal to or less than about 88 cp at the temperature of about 23.6° C., equal to or less than about 87 cp at the temperature of about 23.6° C., equal to or less than about 86 cp at the temperature of about 23.6° C., equal to or less than about 85 cp at the temperature of about 23.6° C., equal to or less than about 84 cp at the temperature of about 23.6° C., equal to or less than about 83 cp at the temperature of about 23.6° C., equal to or less than about 82 cp at the temperature of about 23.6° C., equal to or less than about 81 cp at the temperature of about 23.6° C., equal to or less than about 80 cp at the temperature of about 23.6° C., equal to or less than about 79 cp at the temperature of about 23.6° C., equal to or less than about 78 cp at the temperature of about 23.6° C., equal to or less than about 77 cp at the temperature of about 23.6° C., equal to or less than about 76 cp at the temperature of about 23.6° C., equal to or less than about 75 cp at the temperature of about 23.6° C., equal to or less than about 74 cp at the temperature of about 23.6° C., equal to or less than about 73 cp at the temperature of about 23.6° C., equal to or less than about 72 cp at the temperature of about 23.6° C., equal to or less than about 71 cp at the temperature of about 23.6° C., equal to or less than about 70 cp at the temperature of about 23.6° C., equal to or less than about 69 cp at the temperature of about 23.6° C., equal to or less than about 68 cp at the temperature of about 23.6° C., equal to or less than about 67 cp at the temperature of about 23.6° C., equal to or less than about 66 cp at the temperature of about 23.6° C., equal to or less than about 65 cp at the temperature of about 23.6° C., equal to or less than about 64 cp at the temperature of about 23.6° C., equal to or less than about 63 cp at the temperature of about 23.6° C., equal to or less than about 62 cp at the temperature of about 23.6° C., equal to or less than about 61 cp at the temperature of about 23.6° C., equal to or less than about 60 cp at the temperature of about 23.6° C., equal to or less than about 59 cp at the temperature of about 23.6° C., equal to or less than about 58 cp at the temperature of about 23.6° C., equal to or less than about 57 cp at the temperature of about 23.6° C., equal to or less than about 56 cp at the temperature of about 23.6° C., equal to or less than about 55 cp at the temperature of about 23.6° C., equal to or less than about 54 cp at the temperature of about 23.6° C., equal to or less than about 53 cp at the temperature of about 23.6° C., equal to or less than about 52 cp at the temperature of about 23.6° C., equal to or less than about 51 cp at the temperature of about 23.6° C., equal to or less than about 50 cp at the temperature of about 23.6° C., equal to or less than about 49 cp at the temperature of about 23.6° C., equal to or less than about 48 cp at the temperature of about 23.6° C., equal to or less than about 47 cp at the temperature of about 23.6° C., equal to or less than about 46 cp at the temperature of about 23.6° C., equal to or less than about 45 cp at the temperature of about 23.6° C., equal to or less than about 44 cp at the temperature of about 23.6° C., equal to or less than about 43 cp at the temperature of about 23.6° C., equal to or less than about 42 cp at the temperature of about 23.6° C., equal to or less than about 41 cp at the temperature of about 23.6° C., equal to or less than about 40 cp at the temperature of about 23.6° C., equal to or less than about 39 cp at the temperature of about 23.6° C., equal to or less than about 38 cp at the temperature of about 23.6° C., equal to or less than about 37 cp at the temperature of about 23.6° C., equal to or less than about 36 cp at the temperature of about 23.6° C., equal to or less than about 35 cp at the temperature of about 23.6° C., equal to or less than about 34 cp at the temperature of about 23.6° C., equal to or less than about 33 cp at the temperature of about 23.6° C., equal to or less than about 32 cp at the temperature of about 23.6° C., equal to or less than about 31 cp at the temperature of about 23.6° C., or equal to or less than about 30 cp at the temperature of about 23.6° C.,

In some embodiments, the magnetic ionic liquid disclosed herein is water insoluble, indicated by exhibiting no observable change in color or pH of either the MIL or aqueous phase, or by that the MIL droplets still responded readily to an external magnetic field after three days of suspension in the aqueous phase. In other embodiments, the magnetic ionic liquid disclosed herein has a solubility of less than about 0.01% (v/v) in water. In other embodiments, the magnetic ionic liquid disclosed herein has a solubility of less than about 0.05% (v/v), about 0.04% (v/v), about 0.03% (v/v), about 0.02% (v/v), about 0.009% (v/v), about 0.008% (v/v), about 0.007% (v/v), about 0.006% (v/v), about 0.005% (v/v), about 0.004% (v/v), about 0.003% (v/v), about 0.002% (v/v), about 0.001% (v/v), or any value therein between in water.

In some embodiments, the magnetic ionic liquid disclosed herein has a thermal stability indicated by an onset of decomposition starting at about 110° C. or above. In some embodiments, the magnetic ionic liquid disclosed herein has a thermal stability indicated by an onset of decomposition starting at about 120° C., about 100° C., about 95° C., about 90° C., about 85° C., about 80° C., about 75° C., about 70° C., about 65° C., about 60° C., about 55° C., about 50° C., about 45° C., or any value therein between.

In some embodiments, the magnetic ionic liquid disclosed herein has a magnetic susceptibility from about 2.5μ_{B} to about 10.0μ_{B}, measured by a Quantum Design MPMS SQUID magnetometer. In some embodiments, the magnetic ionic liquid disclosed herein has a magnetic susceptibility from about 0.5μ_{B} to about 3.0μ_{B}, from about 2μ_{B} to about 10μ_{B}, from about 1μ_{B} to about 5μ_{B}, from about 1μ_{B} to about 10.0μ_{B}, from about 2μ_{B} to about 10μ_{B}, from about 3μ_{B} to about 10.0μ_{B}, from about 1μ_{B} to about 5μ_{B}, from about 5μ_{B} to about 10.0μ_{B}, about 10, about 9, about 8, about 7, about 6, about 5, about 4, about 3, about 2, about 1, about 0.5, about 0.2, or any value therein between as measured by a Quantum Design MPMS SQUID magnetometer.

In some embodiments, the magnetic ionic liquid disclosed herein is soluble in hexane, heptane, toluene, and benzene at 10% (v/v) MIL to solvent ratio, in acetone, acetonitrile, chloroform, dichloromethane, dioxane, ethanol, ethyl acetate, diethyl ether, methanol, or isopropyl alcohol at 20% (v/v) MIL to solvent ratio, or in hexane, heptane, toluene, and benzene at 20% (v/v) MIL to solvent ratio.

In some embodiments, the magnetic ionic liquid has a solubility of greater than about 10% (v/v) in an organic solvent (except DMSO). In some other embodiments, the magnetic ionic liquid has a solubility of greater than about 20% (v/v) in an organic solvent (except DMSO). In some other embodiments, the magnetic ionic liquid has a solubility of greater than about 1% (v/v), about 2% (v/v), about 3% (v/v), about 4% (v/v), about 5% (v/v), about 6% (v/v), about 7% (v/v), about 8% (v/v), about 9% (v/v), about 10% (v/v), about 11% (v/v), about 12% (v/v), about 13% (v/v), about 14% (v/v), about 15% (v/v), about 16% (v/v), about 17% (v/v), about 19% (v/v), or any value therein between in an organic solvent (except DMSO).

### Sample

According to the methods disclosed herein, a sample is acquired and/or provided.

A “sample” can be a liquid or solid substance that contains the microbes to be extracted. In some other situation, a sample can one derived from a substance that contains the microbes to be detected through one or more routine water or solvent extraction methods known in the art. In some other situation, a sample can a liquid or solid material that have been used to accumulate microbes from other source. For example, a sample can be a liquid media or solid material that is used to collect aerosols that contain the microbes to be detected via cyclonic concentrator devices.

A sample can be a food sample that is originated directly from animals or plants, such as milk, juice, or derived from those originated from animals or plants, with or without any sample preparation or treatment procedure. A sample can also be a mixture of water or solvent and a specimen collected from any substance that can comprises the microbes to be detected. A sample can be a food sample that is from a food or food source that can comprise microbes.

A sample can a water sample collected from a river, lake, pond, tank, reservoir, surface water, or any other water source. A sample can also be a soil sample taken from any place.

A sample can also be a biological fluid or solid from a mammal or human, such as blood, plasma, urine, feces, or other body fluid or solid.

In some embodiments, the sample is food or a food product. In some other embodiments, the samples is milk, juice, or egg.

In some embodiments, the sample comprises the viable microbes. In some other embodiments, the sample comprises the viable microbes at a concentration of about 10³ CFU/ml or greater.

A sample can be a slurry or aqueous suspension made of a solid or dry food or other solid specimen by dilution and homogenization using water or a buffer system, a common procedure used as the first step in traditional microbiological analysis of solid or dry foods, a surface sample collected into an aqueous medium as a rinsate or from a sponge or swab, or an air sample collected into an aqueous medium via cyclonic concentration.

### Microbes

As disclosed herein, viable microbes are detected, concentrated, and/or extracted from the sample. Microbes can be bacteria, archaea, fungi (yeasts, molds, etc.) protozoa, viruses, or mixtures thereof. Microbes of a specific type (genus, species, etc.) usually share some characteristic DNA or RNA among themselves and can be distinguished from another types of microbes because of their respective characteristic DNA or RNA sequences. Microbes include the family Enterobacteriaceae, which is a related grouping of gram-negative, facultatively anaerobic rod-shaped bacteria. The family contains several genera of importance to agriculture, food safety and human health, including Cronobacter, Enterobacter, Erwinia, Escherichia, Klebsiella, Pantoea, Pectobacterium, Salmonella, Serratia, Shigella and Yersinia. Thus, in some embodiments, the microbes are Cronobacter sakazakii, E. coli, Klebsiella aerogenes, Pantoea eucalypti, Pantoea stewartii, Pectobacterium carotovorum, Salmonella bongori, Salmonella enterica, Serratia marcescens, and/or Yersinia enterocolitica. In some other embodiments, the microbes are yeast for beer production.

In some embodiments, the microbes can be gram-negative bacteria. Various gram-negative are described herein. In other embodiments, the microbes can be gram-positive bacteria.

In some other embodiments, the microbes can be non-pathogenic Escherichia coli, such as E. coli K12 or E. coli ATCC 25922 or related non-pathogenic bacteria such as Serratia marcescens. In yet some other embodiments, the microbes can pathogenic Escherichia coli, such as Escherichia coli O157:H7, an important foodborne pathogen considered as an adulterant if present in foods.

In some embodiments, the microbes can be Salmonella enterica, Salmonella bongori, or a combination of Salmonella enterica and Salmonella bongori. In some embodiments, the microbes are Salmonella having a truncated outer membrane. In some other embodiments, the microbes are Salmonella Minnesota.

Gram-negative bacteria, such as members of the Family Enterobacteriaceae, possess a protective outer barrier layer, called the outer membrane (OM). The OM serves to limit the passive diffusion of molecules, particularly hydrophobic molecules (such as certain classes of antibiotics) into the interior of the cell. Treatment or interaction with various chemicals may physically damage the OM and other cellular structures or components, causing what is broadly characterized as “cell injury”.

Cell injury can be detected by plating chemically-treated cells in parallel on both non-selective and selective agars and evaluating growth under each condition. Physiologically healthy (non-injured) gram-negative cells can tolerate exposure to selective agents-compounds such as the dye crystal violet or sodium desoxy cholate, which are inherently toxic to gram-positive cells, which do not possess an OM structure. Injury to gram-negative cells caused by exposure to deleterious chemicals (or other “insults” such as heat) is typically characterized by damage to the OM, causing the cell to become “leaky”, allowing ingress of these selective/toxic compounds, resulting in reduced growth on selective agars. Injury can therefore be detected by plating treated cells in parallel on both non-selective and selective agars and comparing the results. For example, when we exposed suspensions of S. Typhimurium to the Ni(II) MIL for times ranging from 0 min (essentially our standard 30 s extraction protocol) to 15 min, the resulting average CFU counts on TSA and BSA appeared similar, regardless of exposure time. We explored these data further using the statistical analyses reported in the Electronic Supplementary Material. For TSA, no statistically significant differences were shown in the CFU counts over time. For cases that were plated to BSA, we did find significant evidence that the CFU counts at time 5 min, 10 min, and 15 min are statistically greater compared to time 0 counts. While not wishing to be bound by the theory, we hypothesize that if the Ni(II) MIL was chemically injurious to S. Typhimurium, counts on BSA would be lower than those observed on TSA, and further, that BSA counts would continue to fall as a function of exposure time. Although we found that CFU counts on BSA were statistically different from counts at time zero, the observation that counts at 5, 10 and 15 min were greater than those at time zero support our conclusion that the Ni(II) MIL did not cause detectable injury to S. Typhimurium with this media pairing, even after exposure periods thirty times longer than our standard 30 s extraction time.

If chemically-treated cells grow to the same or similar degree on non-selective and selective agars, this indicates that the chemical treatment did not cause cellular injury and the chemical is not inherently toxic to the cells. Alternatively, if chemically-treated cells show a differential between growth on non-selective and selective agars, with lower growth on the selective agar, the chemical treatment did cause cellular injury and was toxic to the cells. In our toxicity testing, the “chemical” used to treat bacteria was the Ni(II) MIL.

Multiple strains of Salmonella and E. coli O157:H7 were tested for injury after exposed to the exemplary MIL disclosed herein for periods corresponding to the contact time experienced during our normal MIL capture and concentration protocol (as well as for longer periods of time), then plated in parallel on both non-selective and selective agars. The results showed that the exemplary MILs did not show toxicity towards wildtype Salmonella and E. coli O157:H7 strains.

It was surprising or unexpected to find out that the disclosed method was able to capture a “deep rough” mutant of Salmonella enterica serovar Minnesota, which has a defect in its OM and a severely truncated lipopolysaccharide (LPS) layer. Cells with truncated out LPS/OM are often dramatically more susceptible to chemically harsh environments or antimicrobial agents than are cells with intact LPS/OM, as they no longer possess this important barrier structure.

The claimed method's ability to capture this “deep rough” strain demonstrates two important advantages of the claimed method: 1) The capacity of the MILs to effectively bind to (capture) and concentrate these important gram-negative variants that display a very different surface to the external environment and 2) The post-capture growth behavior of this physiologically sensitive strain indicates that the claimed method is not physically harsh or deleterious to the microbes. The lack of toxic impact of the claimed method on the “deep rough” S. Minnesota strain could result from a lack of inherent chemical toxicity of the MIL, from the minimal diffusivity of the hydrophobic MIL in liquid media (which would be expected to prevent MIL molecules from entering the cell), or a combination of both of these possibilities.

In some other embodiments, the microbes can be other pathogenic bacteria. In some other embodiments, the microbes are Cronobacter, problematic bacteria in dried milk foods, including infant formula. In other embodiments, the microbes are Erwinia, an economically important plant pathogen causing vegetable soft rot. In yet some other embodiments, the microbes are Klebsiella, clinically important antibiotic-resistant strains. In some other embodiments, the microbes are Shigella or Yersinia, genera that contain important food pathogens. In some other embodiments, the microbes are Y. pestis, a causative agent of bubonic plague.

In some embodiments, during or after the contacting step, any manual or mechanical method can be used during the contact time to maximize the mixing of the MIL and the sample. In some embodiments, vortexing is used. In some embodiments, mechanical stirring is used.

In some embodiments, during or after the contacting step or during the extracting step, the sample or the mixture of the sample and MIL may subject to heat, ultrasound, pressure, water, solvent, or chemical treatment, or a combination thereof, in order to increase the extraction of the microbes and/or their diagnostically useful cellular components, including, but not limited to DNA, RNA, other nucleic acids, proteins, enzymes, lipids or cell wall materials characteristic of the target organism.

In some embodiments, during or after the contacting step, the microbes from the sample stay intact or remain viable in the MIL. In some embodiments, during or after contacting step, the microbes in MIL become disintegrated or lysed to generate their diagnostically useful cellular components, including, but not limited to DNA, RNA, other nucleic acids, proteins, enzymes, lipids or cell wall materials characteristic of the target organism. In some embodiments, the MIL or the claimed method does not cause injury to cells of the microbes.

In some embodiments, after the “contacting” step and before the “extracting” step, the MIL comprises the viable microbes, diagnostically useful cellular components, including, but not limited to DNA, RNA, other nucleic acids, proteins, enzymes, lipids or cell wall materials characteristic of the target organism, or a combination thereof. In some embodiments, the MIL comprises viable microbes. In some other embodiments, the MIL comprises lysed cells of the microbes. In yet some other embodiments, the MIL comprises the characteristic or unique DNA or RNA of the microbes.

As used herein, “using Recombinase Polymerase Amplification (RPA) on the extracted sample for amplifying DNA or RNA of the microbes” refers to contact an enzyme for Recombinase Polymerase Amplification (RPA) and a primer or primer set with the extracted sample for the purpose of amplifying the characteristic DNA or RNA of the microbes. In some embodiments, an additional reverse transcriptase (RT) enzyme is used to contact the extracted sample if the characteristic RNA sequence is targeted for amplification. In some embodiments, the primer or primer set is for the dienelactone hydrolase or “DLH” enzyme of Salmonella. In some other embodiments, the primer or primer set for genes related to innate bacterial immunity mechanisms (CRISPR sequences) unique to the microbes.

The microbes' characteristic DNA or RNA, which is amplified in the subsequent Recombinase Polymerase Amplification (RPA), includes virulence factors or other DNA or RNA sequences not involved in virulence but otherwise unique to the target microbes. Many of these characteristic or unique DNA or RNA sequences are known for specific target organisms. Others can be discovered via methods such as comparative genomics, or analysis of CRISPR sequences.

To achieve specific detection of an organism, we can target its DNA or RNA, as long as the sequence is unique to a specific microbe. In some embodiments, the characteristic RNA sequence of the microbes is amplified using reverse transcription RPA, an RPA reaction to which the enzyme reverse transcriptase is added in order to create amplifiable DNA from RNA, as described below. The advantage of utilizing the characteristic RNA, such as ribosomal RNA, is that such RNA sequence exists in multicopy.

In some embodiments, the characteristic DNA or RNA sequence is a gene or sequence associated with a virulence factor for a pathogen. One such exemplary gene is the invasion protein gene invA that can used for NALFIA-based detection of Salmonella. The other genes for virulence factors can include those for bacterial flagella involved in initial attachment of bacteria to host cells or for additional proteins involved in bacterial virulence, such as the outer membrane protein OmpA found in various gram-negative bacilli.

In other cases, the characteristic DNA or RNA sequences are genes or sequences that are not associated with virulence, but are present only in the microbe being tested for. Examples of these genes include the putative dienelactone hydrolase gene that was determined to be restricted to Salmonella through comparison of the Salmonella genome against the genomes of several closely-related bacteria, or other genetic elements, such as Clustered Regularly Interspaced Short Palendromic Repeats (CRISPR) sequences. In these cases, the ultimate function of the gene or sequence is not important, as long as it is unique to the microbe that is tested for. In some embodiments, the characteristic DNA or RNA sequences are genes or parts of genes involved in bacterial metabolism (enzymes, such as the dienelactone hydrolase or “DLH” enzyme) or genes related to innate bacterial immunity mechanisms (CRISPR sequences).

A characteristic or unique DNA or RNA sequences for a microbe can be determined or verified experimentally using assays with target and non-target strains. Alternatively, a characteristic or unique DNA or RNA sequences for a microbe can be determined in silico (computer-based) screening of data, using the PrimerBlast tool from the National Center for Biotechnology Information (NCBI). This tool allows one to screen large DNA databases to check the exclusivity (does it only detect the pathogen you're looking for?) and inclusivity (how many strains or species of this pathogen does it detect?) of the sequence targeted by the assay.

In some embodiments, using a characteristic RNA for RPA is preferred, especially ribosomal RNA (rRNA). rRNA contains variable regions that contain pathogen-specific sequences. Additionally, rRNA is present in cells at high copy numbers-up to 100,000 copies, instead of a single copy as is the case with some genomic targets. High target copy numbers can significantly enhance the sensitivity of an amplification assay such as RPA.

However, because RPA is a method for DNA amplification, RPA assays must be modified to allow detection of RNA. Specifically, a reverse transcriptase (RT) enzyme must be included for characteristic RNA detection. The RT enzyme will generate complementary DNA (cDNA) from the single-stranded rRNA template and the RPA enzyme system can use this cDNA as a substrate for DNA amplification.

Various virulence genes and virulence-enhancing genes and their corresponding primer sets have been investigated and described for the polymerase chain reaction (PCR) to detect and characterize Salmonella, as in Chapter 4, The Use of Molecular Methods for Detecting and Discriminating Food Borne Infectious Bacteria, Levin, R. E., CRC, 2010, CRC Press which is hereby incorporated by reference. There are presently over 30 Salmonella-specific genes that have been used for Salmonella. These include invA gene sequences that are highly conserved among all Salmonella serotypes.

A general principle, procedure, and optimization for DNA/RNA amplification using recombinase polymerase amplification (RPA) has been described, as in TwistAmp® DNA Amplification Kits, Assay Design Manual (https://www.twistdx.co.uk/docs/default-source/RPA-assay-design/twistamp-assay-design-manual-v2-5.pdf), which is hereby incorporated by references.

According to the TwistAmp® manual, “Primers designed for a given PCR assay may often work in RPA but may not be optimal for TwistAmp® reactions. For assays requiring less stringent detection (greater than 1.000 copies per sample tested) only a small number of primers need be screened in most cases and use of PCR primers may be sufficient.” In other words, DNA/RNA sequences and their corresponding primer sets reported in the literature for PCR-based detection of Salmonella are helpful as a starting point for detection of Salmonella using RPA, but success of using the same in RPA is not predicable, considering the interference from different samples and their matrixes can be different for PCR and RPA, let alone in the claimed methods using both MIL and RPA.

Indeed, as indicated in this disclosure, targeting genes or DNA/RNA sequences with the corresponding primer sets recommended for PCR may not generate the optimal results for the disclosed methods that use RPA and/or MIL. For examples, the DLH primer set, despite its apparent divergence from existing RPA primer design considerations, was found to be surprisingly more useful for RPA and the claimed methods, compared to the InvA gene and its corresponding primer sets preferred for PCR.

In some embodiments, during the extracting step, the microbes in the MIL and/or the extracting medium stay intact or remain viable. In some embodiments, during the extracting step, the microbes become disintegrated or lysed to generate their diagnostically useful cellular components, including, but not limited to DNA, RNA, other nucleic acids, proteins, enzymes, lipids or cell wall materials characteristic of the target organism.

As used herein, an extracting medium refers to an aqueous medium or solution whose ionic strength is adjusted through the addition of various ionic species. In some embodiments, the aqueous medium promotes the release of the captured cells (microbes or diagnostically useful cellular components, including, but not limited to DNA, RNA, other nucleic acids, proteins, enzymes, lipids or cell wall materials characteristic of the target organism) from the MIL. In some embodiments, the extracting medium comprises tryptone, yeast extract, sodium chloride or a combination thereof as the ionic species used to adjust the ionic strength.

In some embodiments, the extracting medium does not affect cell viability. In some other embodiments, the extracting medium promotes cell release, cell lysis, microbes' release of their characteristic DNA/RNA for analysis, or a combination thereof, compared to using water alone. In some embodiments, the aqueous extracting medium enhances the microbes' or their diagnostically useful cellular components', including, but not limited to DNA, RNA, other nucleic acids, proteins, enzymes, lipids or cell wall materials characteristic of the target organism, recovery, compared to using water.

In some embodiments, the extracting medium is a Luria-Bertani-derived nutrient broth. In some other embodiments, the extracting medium is an aqueous solution comprising yeast extract for microbiology. In some other embodiments, the extracting medium is a Luria-Bertani-derived nutrient broth comprising tryptone. In yet some other embodiments, the extracting medium is a Luria-Bertani-derived nutrient broth comprising yeast extract for microbiology. In some embodiments, the extracting medium is a Luria-Bertani-derived nutrient broth comprising NaCl. In some other embodiments, the extracting medium is a Luria-Bertani-derived nutrient broth comprising one or more of tryptone, yeast extract, and NaCl.

In some embodiments, the extracting medium is a Luria-Bertani-derived nutrient broth or aqueous solution comprising from about 1 g/L to about 30 g/L, from about 1 g/L to about 25 g/L, from about 1 g/L to about 20 g/L, from about 1 g/L to about 15 g/L, from about 1 g/L to about 10 g/L, about 2, about 4, about 6, about 8, about 10, about 12, about 14, about 16, about 18, about 20, about 22, about 24, about 26, about 28, or any value there between of one or more tryptone, yeast extract, NaCl, or other salt(s) that are routinely used in microbiology for changing ionic strength of an aqueous solution.

In some embodiments, the extracting medium is a Luria-Bertani-derived nutrient broth or aqueous solution comprising from about 1 g/L to about 20 g/L, from about 1 g/L to about 15 g/L, from about 1 g/L to about 10 g/L of tryptone and from about 1 g/L to 10 g/L or from about 1 g/L to about 5 g/L of yeast extract.

In some other embodiments, the extracting medium is a Luria-Bertani-derived nutrient broth or aqueous solution comprising from about 1 g/L to about 20 g/L, from about 1 g/L to about 15 g/L, from about 1 g/L to about 10 g/L of tryptone and from about 1 g/L to 10 g/L or from about 1 g/L to about 5 g/L of NaCl.

In some embodiments, wherein the extracting medium is a Luria-Bertani-derived nutrient broth comprising more than 10 g/L of tryptone, more than 5 g/L of yeast extract, more than 10 g/L of NaCl, or combination thereof. In some other embodiments, the extracting medium is a Luria-Bertani-derived nutrient broth comprising from more than 10 g/L to about 20 g/L of NaCl, from more than 10 g/L g/L to about 20 g/L of tryptone, from more than 5 g/L to about 20 g/L of yeast extract, or combination thereof.

In some embodiments, the power-free heat source is a chemical heat pack. A chemical heat pack is based on exothermic chemical reactions and may be regenerable. In some other embodiments, the power-free heat source is a portable heat source energized by a battery or solar energy or light. In some other embodiments, the power-free heat source is a USB-powered incubator or battery powered infrared lamp. In some other embodiments, the power-free heat source is body heat from a mammal or human.

During the contacting time, the microbes, their diagnostically useful cellular components, including, but not limited to DNA, RNA, other nucleic acids, proteins, enzymes, lipids or cell wall materials characteristic of the target organism, or both migrate from the sample to the MIL. In some embodiments, the magnetic ionic liquid extracts the viable or intact microbes from the sample during the contact time. In some other embodiments, the MIL extracts the microbes' diagnostically useful cellular components, including, but not limited to DNA, RNA, other nucleic acids, proteins, enzymes, lipids or cell wall materials characteristic of the target organism, from the sample during the contact time.

In some other embodiments, wherein the medium enhances the microbes' or their diagnostically useful cellular components', including, but not limited to DNA, RNA, other nucleic acids, proteins, enzymes, lipids or cell wall materials characteristic of the target organism, recovery, compared to using water.

In some embodiments, a volume ratio between the magnetic ionic liquid and the extracting medium is from about 1:5 to about 1:15, from about 5:1 to about 1:15, from about 2:1 to about 1:15, from about 1:1 to about 1:15, from about 1:3 to about 1:15, from about 1:5 to about 1:10, from about 1:5 to about 1:8, or any value there between.

In some embodiments, the extracted microbe sample comprises the viable microbes, diagnostically useful cellular components, including, but not limited to DNA, RNA, other nucleic acids, proteins, enzymes, lipids or cell wall materials characteristic of the target organism or a combination thereof. In some embodiments, the extracted microbe sample comprises the viable microbes. In some other embodiments, the extracted microbe sample comprises the lysed cell of the microbes. In yet some other embodiments, the extracted microbe sample comprises the characteristic or unique DNA or RNA of the microbes.

In some embodiments, the method further comprises lysing the microbes' cells in the extracted sample. The method for lysing cell can be heat, chemical lysing, electrical shock, or any other method known in the art.

In some embodiments, the cells are subject to diagnostics for detection. The cells can be subject to amplification or non-amplification-based diagnostics.

In some embodiments, the magnetic ionic liquid disclosed herein can extract viable microbes from a sample comprising the microbes. In some embodiments, the magnetic ionic liquid disclosed herein can preconcentrate viable microbes or their diagnostically useful cellular components, including, but not limited to DNA, RNA, other nucleic acids, proteins, enzymes, lipids or cell wall materials characteristic of the target organism, from a sample comprising the microbes. As used herein, “preconcentrate” means that the viable microbes or their diagnostically useful cellular components, including, but not limited to DNA, RNA, other nucleic acids, proteins, enzymes, lipids or cell wall materials characteristic of the target organism, has a higher concentration in the MIL than in the sample after the MIL is mixed with the sample.

In some embodiments, for the method of extracting, concentrating, detecting viable microbes, the magnetic ionic liquid has a higher concentration of the microbes or their diagnostically useful cellular components, including, but not limited to DNA, RNA, other nucleic acids, proteins, enzymes, lipids or cell wall materials characteristic of the target organism, than the sample, after the contacting step. In some embodiments, the ratio of the microbe concentration in the magnetic ionic liquid to one in the sample is from about 1:1 to about 50:1. In some embodiments, the ratio of the microbe concentration in the magnetic ionic liquid to one in the sample is from about 1:1 to about 25:1, from about 1:1 to about 20:1, from about 1:1 to about 15:1, from about 1:1 to about 10:1, or from about 1:1 to about 5:1. In some embodiments, the microbe or its diagnostically useful cellular components, including, but not limited to DNA, RNA, other nucleic acids, proteins, enzymes, lipids or cell wall materials characteristic of the target organism, concentration in the magnetic ionic liquid is higher than one in the sample after the contact time. In some embodiments, the microbe concentration in the magnetic ionic liquid can be lower than in the sample after the contact time.

In some embodiments, the ratio of the microbe concentration in the magnetic ionic liquid to one in the sample is from about 1:1 to about 2:1, from about 1:1 to about 40:1, about 1:1, about 5:1, about 10:1, about 15:1, about 20:1, about 25:1, about 30:1, about 35:1, about 40:1, about 50:1, or any value therein between.

In some embodiments, for the method of extracting, concentrating, detecting viable microbes, the magnetic ionic liquid has a lower concentration of the microbes or their diagnostically useful cellular components, including, but not limited to DNA, RNA, other nucleic acids, proteins, enzymes, lipids or cell wall materials characteristic of the target organism, than the sample, after the contacting step.

In some embodiments, for the method of extracting, concentrating, detecting viable microbes, the weight ratio between the magnetic ionic liquid and the sample is between about 1:10 to about 1:100. In some embodiments, for the method of extracting, concentrating, detecting microbes, the weight ratio between the magnetic ionic liquid and the sample is between about 1:10 to about 1:20, between about 1:10 to about 1:30, between about 1:10 to 1:40, between about 1:10 to 1:50, between about 1:10 to 1:60, between about 1:70 to 1:80, between about 1:10 to 1:90, between about 10:1 to 1:10, between about 1:20 to 1:50, between about 1:20 to 1:100, between about 1:40 to 1:80, between about 1:50 to 1:10, between about 1:10 to 1:20, about 10:1, about 5:1, about 1:1, about 1:5, about 1:10, about 1:20, about 1:30, about 1:40, about 1:50, about 1:60, about 1:70, about 1:80, about 1:90, about 1:100, or any value therein between.

In some embodiments, for the method of extracting, concentrating, detecting microbes, the viable microbes have a concentration of at least 10³ or 10⁴ CFU/mL in the sample or liquid part of the sample.

In some embodiments, for the method of extracting, concentrating, detecting microbes, wherein the sample is a heterogeneous aqueous solution. In some other embodiments, the sample is a heterogeneous aqueous solution comprising food, milk, juice, biological fluid, blood, or any suspended food solid. In yet some other embodiments, the sample is an aqueous solution comprising milk, juice, biological fluid, or blood. In some other embodiments, the sample is an aqueous solution comprising or suspended with any material that can host viable microbes. In yet some other embodiments, the sample comprises soil that can host the microbes.

In some embodiments, for the method of extracting, concentrating, detecting microbes, the contact time for the extracting step is from about 30 seconds to about 10 min. In some embodiments, the contact time is from about 1 minute to about 1 hour, from 1 minute to about 2 hours, from 1 minute to about 5 hours, from about 1 hour to 24 hours, about 5 minutes, about 10 minutes, about 2 minutes, about 1 hour, about 2 hours, about 5 hours, about 10 hours, or any value therein between.

In some embodiments, during the contact time, a manual or mechanical method is utilized to maximize the contact between the MIL and the food sample for the whole contact time or only for a part of the contact time.

In some embodiments, the method of extracting, concentrating, detecting microbes further comprises mixing the sample and magnetic ionic liquid during contact time through manual or mechanical agitation after the contacting step starts. Vortexing and hand shaking are examples of agitation to maximize the interaction between the MIL and the sample.

In some embodiments, the method of extracting, concentrating, detecting microbes further comprises separating the magnetic ionic liquid from the sample by a magnetic field. In some other embodiments, the method further comprises separating the magnetic ionic liquid from the sample by a magnetic field of from about 0.1 tesla to about 2 tesla.

In some embodiments, the method of extracting, concentrating, detecting microbes comprises extracting the microbes or their diagnostically useful cellular components, including, but not limited to DNA, RNA, other nucleic acids, proteins, enzymes, lipids or cell wall materials characteristic of the target organism, from the magnetic ionic liquid to an aqueous extracting medium. In some embodiments, the aqueous extracting medium is a nutrient broth, salt solution, or aqueous medium that recovers the microbes or their diagnostically useful cellular components, including, but not limited to DNA, RNA, other nucleic acids, proteins, enzymes, lipids or cell wall materials characteristic of the target organism, from the MIL.

In some embodiments, the method of extracting, concentrating, detecting microbes further comprises enriching, culturing, or multiplying the microbes extracted from the sample by the MIL and/or the extracting medium. The techniques are any one of those that would be used by one skilled in the art to increase population of microbes.

In some other embodiments, the method further comprises using mass/flow cytometry for detecting characteristic DNA of the microbes. In yet some other embodiments, the method further comprises using a culture-based method to multiply and identify the microbes.

### Preanalytical Sample Preparation

Detection of bacterial pathogens is a multi-step process preferably comprised of 1) a sampling step. 2) a sample-preparation step and 3) a detection step. Sampling involves application of well-established, statistically-validated approaches for product-specific analysis, prescribing the sampling procedures and number of samples needed to detect a certain level of microbes (if present) with a given degree of certainty. Sample preparation refers to the various procedures needed to process the raw sample so that it is amenable to further testing. In the case of microbial diagnostics, this involves various physical, chemical or combination treatments aimed at performing key functions, such as producing of a homogeneous sample, reducing sample volume, excluding or inactivating inhibitory substances, separating cells from food (or environmental or clinical) matrices, concentrating these cells and purifying them (removal of cell-associated assay inhibitors). Detection involves use of reagents and instruments for direct detection of organism-associated analytes (specific DNA or RNA sequences or other diagnostically-important cell features, such as unique proteins, enzymes, antigens, etc.). When proceeding from sample-to-answer, it is important to understand that inefficiencies at any of these independent, yet interdependent steps (sampling, sample preparation, detection) will propagate through the chain and impact the final results.

It is important to note that a single sample preparation step may be compatible with multiple downstream detection steps. For example, a short, slow centrifugation may be used as a means for selective sedimentation of large food particulates, while retaining the smaller and lighter bacterial cells in the supernatant. These supernatant-associated cells may then either be tested directly or subjected to further sample preparation, such as filtration, immunomagnetic separation or longer, faster centrifugation for concentration of bacterial cells (Brehm-Stecher et al., 2009).

We have found that use of MILs can provide a general, yet customizable platform for bacterial capture and concentration. Sample preparation ultimately allows a great deal of flexibility in assay construction. Our described use of MILs for preanalytical sample preparation of foods (Hice et al., 2019) accomplishes several of the ideal objectives of preanalytical sample preparation, including (capturing and) concentrating cells from the test matrix, purifying them, reducing the sample volume and producing a homogeneous sample. As MIL-based sample preparation is an independent step or module, the cells processed in this manner can be analyzed using any number of downstream detection approaches, which we categorize and describe further as “Amplification-Based Diagnostics” and “Non-Amplification-Based Diagnostics”.

### Amplification-Based Diagnostics

Amplification-based diagnostics are those that result in an enrichment of diagnostically-important biomolecules to levels where they can be easily detected. Example target molecules include nucleic acids (NA) such as DNA and RNA. An example of an amplification-based diagnostic test is the polymerase chain reaction (PCR) or its variants. With PCR, even a single pathogen-specific NA sequence can be exponentially enriched to a detectable level with high specificity and from complex samples containing high levels of non-target bacteria. Detection of an amplified NA sequence (an amplicon) by PCR provides evidence that the sequence, and therefore the pathogen of interest, was in the original sample. PCR depends on temperature cycling between denaturation, annealing and extension steps, or in abbreviated processes, denaturation and a combined annealing/extension step, which can provide efficiencies in speed, as it reduces cycling time. Certain advances, such as convective PCR (Krishnan et al., 2002) can accomplish temperature cycling by passage of a bolus of reagents through a temperature zones corresponding to denaturation and annealing/extension temperatures, but most commercial PCR applications remain dependent on specialized, dedicated cycling instruments.

An exciting development in nucleic acid diagnostics is the advent of isothermal amplification methods. The term “isothermal” refers to a process that occurs at a single, constant temperature. Isothermal NA amplification steps mimic cellular processes, which in higher organisms occur within temperature-regulated tissues and in bacteria at temperatures associated with the medium (water, food, etc.) in which the bacteria are growing. Isothermal NA amplification simplifies the equipment needed to generate pathogen-specific amplicons and are not limited by instrumental factors such as temperature cycling speed, allowing target organisms to be quickly identified in a sample. Several methods have been described for isothermal amplification of NA, these include, but are not limited to, transcription-mediate amplification (TMA), self-sustained sequence replication (3SR), nucleic acid sequence-based amplification (NASBA), signal mediated amplification of RNA technology (SMART), strand displacement amplification (SDA), rolling circle amplification (RCA), loop-mediated isothermal amplification of DNA (LAMP), isothermal multiple displacement amplification (IMDA), helicase-dependent amplification (HDA), single primer isothermal amplification (SPIA), circular helicase-dependent amplification (cHDA) and recombinase polymerase amplification (RPA) (Gill and Ghaemi, 2003; Hice et al., 2019). Hice et al., (2019) coupled MIL-based capture and concentration of S. Typhimurium to RPA, using inexpensive and regenerable sodium acetate heat packs to drive RPA reactions, eliminating the need for an external power source. Salmonella RPA amplicons could be detected in <10 min using a simple chromatographic readout. This approach highlights the utility of MIL-based sample preparation in conjunction with isothermal NA amplification to provide a simple, streamlined detection method that is amenable to analyses in the field or in other resource-limited environments.

Preferably the amplification-based diagnostic is performed at a temperature of from about 20° C. to about 60° C., more preferably from about 20° C. to about 55° C., still more preferably from about 20° C. to about 50° C., even more preferably from about 25° C. to about 45° C., and most preferably from about 25° C. to about 40° C.

### Non-Amplification-Based Diagnostics

Non-amplification-based diagnostics are those approaches that do not depend on targeted biomolecule amplification for detection of target organisms. This class of diagnostics is very broad and includes culture-based detection of bacteria using either non-selective or selective agars, labeling with fluorescent antibodies, fluorescence in situ hybridization (FISH) using RNA, DNA, peptide nucleic acid (PNA), locked nucleic acid (LNA) or other natural or synthetic probes capable of binding to NA via Watson-Crick pairing, spectroscopic methods including vibrational spectroscopy and mass spectroscopy, and others.

Another non-amplification-based diagnostic method is Gram-staining, which represents the first fundamental step towards identifying bacteria using classical bacteriological techniques. In the Gram staining process, unknown bacteria are heat fixed then exposed to a sequence of individual staining steps involving 1) crystal violet dye, 2) iodine, 3) a solvent (ethanol or acetone) and 4) safranin counterstain. Following this procedure, Gram observed that some bacteria retained crystal violet dye while some did not. Crystal violet-stained cells (purple) are referred to as “gram-positive”. Cells that do not retain crystal violet are counterstained (pink) with safranin and are referred to as “gram-negative”.

Many years later, it was determined that this differential staining is due to fundamental structural differences between the cell envelopes of these two classes of bacteria, with gram-positive bacteria having a single phospholipid membrane and a thick peptidoglycan layer in their cell wall which traps a crystal violet-iodine complex, preventing its removal during the solvent-based decolorization. Conversely, gram-negative bacteria have a double membrane structure and a much thinner peptidoglycan layer that does not retain the complex.

Although gram-positive and gram-negative bacteria differ in gross structure and members of the two bacterial lineages are physiologically and genetically diverse, their surfaces are adorned with similar classes of macromolecules, including polysaccharides and proteins. These macromolecules possess carboxylate, amino and phosphate moieties whose ionization state is dependent on environmental pH (Wilson et al., 2001). The molecular complexity of bacterial cells therefore gives rise to a commensurately complex distribution of surface charge. While the net surface charge of bacteria is negative, individual charged residues can interact, partner and bind with charged solutes (salts, peptides, nucleic acids, etc.) or with suspended molecular aggregates (solid particles such as cationic beads, metal hydroxides such as zirconium hydroxide, minerals such as hydroxyapatite, or liquid aggregates comprised of hydrophobic liquids such as magnetic ionic liquids).

Magnetic ionic liquids (MILs) are paramagnetic molten salts comprised of organic/inorganic cations and anions that exhibit melting points at or below about 100° C. In their use as bacterial capture reagents, MILs are added to aqueous sample suspensions and the suspension is vortexed thoroughly to form MIL microdroplets which then collide with bacteria in the sample. When MIL droplets collide with bacterial cells, we theorize that cationic and anionic MIL partners are able to interact with charged species on cell surfaces, facilitating electrostatic capture of the cells. Loss of coordinating ligands in the anionic component of the MIL may also occur, leaving the cationic metal free to interact with anionic species on bacterial surfaces, which could also promote cell capture. MIL interaction with bacterial cell surfaces is hypothesized to be driven by equilibrium theory, and factors such as strain-specific charge variability and the pH and ionic composition of the sample suspension medium are expected to promote or reduce binding.

Because the MILs are hydrophobic and denser than water, they eventually coalesce at the bottom of an aqueous food sample, a process which can be accelerated through use of an externally applied magnetic field. Consolidation of MIL droplet-bacterial cell complexes into a physically separated liquid phase leads to concentration of captured bacteria, which can then be eluted from the MIL surface by adding an ionically-rich liquid medium that favors displacement of MIL species, resulting in release of cells for downstream processing.

Structural components contributing to cell surface charge in gram-negative bacteria include the lipopolysaccharide outer membrane, a portion of which is referred to as the “O-antigen”, polysaccharide capsular material in mucoid variants or in species forming capsule-enclosed aggregates of cells (“symplasmata”, as with Pantoea eucalypti, which forms these multicellular structures containing hundreds of clonal cells bound within a thick polysaccharide envelope) and structures such as the repeat polysaccharide sequence known as the Enterobacterial Common Antigen (Octavia and Lan, 2014). Additional cell surface structures that contribute to the molecular and charge diversity of gram-negative bacteria include transmembrane proteins, fimbriae (also referred to as adhesins or pili)—stiff, hair-like appendages uniformly distributed across the cell surface and that mediate bacterial binding to host cells, two-dimensional, surface-associated protein arrays (S-layers) and flagella. Flagella (H-antigen) are whip-like structures that confer cell motility and whose number and surface arrangement may vary according to cell type (Octavia and Lan, 2014).

Structural components contributing to cell charge in gram-positive bacteria include those previously mentioned for gram-negative bacteria and include capsular polysaccharides, surface proteins, fimbriae (also called adhesins or pili), S-layers and flagella (Corbett et al., 2010; Melville and Craig, 2013; Messner et al., 2010). gram-positive bacteria also display teichoic acids (TA), which fortify cell wall rigidity by complexing with metal ions, including magnesium and sodium (Rajagopal and Walker, 2017). TA anchored to cell membrane lipids are referred to as “lipoteichoic acids”. Those attached covalently to cell wall peptidoglycan are referred to as “cell wall teichoic acids”. D-alanine ester or D-glucosamine modification of cell wall TA can impart zwitterionic character to these molecules (Kohler et al., 2009; Rautenberg et al., 2010).

Considering the information provided above, both gram-negative and gram-positive bacteria possess and display similar classes of macromolecules at their surfaces. Despite expected differences in polysaccharide or protein sequences among different bacteria, these macromolecules are expected to behave similarly, acting as key mediators of overall cell charge and serving as substrates for partitioning of MIL components, resulting in electrostatic binding and capture of cells. By adjusting the ionic environment, the binding equilibrium can be shifted to promote elution of captured cells back into aqueous suspension for downstream analysis.

### Sample Preparation

While no preanalytical sample preparation method can be considered universal, it is reasonable to expect, by those skilled in the art of bacterial detection techniques, that MIL-based capture and concentration of bacteria can be effectively coupled with many different types of detection techniques, beyond what we have already reduced to practice (qPCR, RPA, non-selective agars, selective agars).

After sample preparation, collected bacteria cells can be subjected to direct visual detection via optical methods such as microscopy or flow cytometry, with or without application of macromolecule-specific chemical stains (stains indicating the presence of DNA, RNA, proteins, lipids, carbohydrates, etc.). Alternatively, cells can be processed for whole-cell molecular detection using rRNA-targeted fluorescent probes. These same cells may also be processed further via heat and/or chemical treatment for fractional analysis of diagnostic cellular components such as nucleic acids, using amplification-based methods such as PCR or chemically-enhanced approaches such as Enzyme-Linked Immunosorbent Assay (ELISA). These examples highlight the inherent generality of many preanalytical sample preparation techniques, where a single method for capture and concentration of bacterial cells from a sample may provide output that is suitable for multiple downstream testing methods.

### RPA

In some embodiments, the RPA is applied to the extracted microbe sample for amplifying characteristic DNA or RNA of the microbes at a temperature of from about 20° C. to about 50° C., from about 20° C. to about 25° C., from about 20° C. to about 30° C., from about 20° C. to about 35° C., from about 20° C. to about 40° C., from about 20° C. to about 45° C., from about 25° C. to about 50° C., from about 25° C. to about 45° C., from about 25° C. to about 40° C., from about 25° C. to about 35° C., from about 30° C. to about 50° C., from about 30° C. to about 45° C., from about 35° C. to about 50° C., from about 35° C. to about 45° C., from about 37° C. to about 42° C., or about values there between.

In some embodiments, wherein the microbes are Salmonella and the RPA comprises using a DLH primer. In some other embodiments, the microbes are Salmonella and the RPA comprises using an invA primer.

In some embodiments, “using Recombinase Polymerase Amplification (RPA) on the extracted sample for amplifying DNA or RNA of the microbes” comprises using a power-free-heat source for RPA. As used herein, “a power-free-heat source” means a tool or device that can provide heat for RPA to be carried out at a temperature or temperature range, without using electricity at the same time. A power-free-heat source may be a battery-powered heating device (heating pad, adjustable hot plate, infrared heating lamp, etc.) or any device utilizing chemical reaction(s), solar energy, or light to generate heat, or any device capable of maintaining an appropriate RPA incubation temperature without using electricity.

In some embodiments, the claimed method comprises using a battery-powered heat source for RPA. In some other embodiments, the claimed method comprises using a chemical heat source for RPA.

The existence or absence of the amplified DNA or RNA sequences after RPA with specific primer(s) or primer set(s), which should be unique or characteristic for specific microbe(s), can be detected or confirmed by a proper and routine tool or device known to one skilled in the art for amplicon detection.

In some embodiments, the method disclosed herein further comprising detecting the amplified DNA using an amplicon detection tool or device. In some embodiments, the detection tool or device is gel electrophoresis. In some other embodiments, the detection tool or device is a nucleic acid lateral flow immunoassay (NALFIA).

Detection of pathogen-specific amplicons via NALFIA uses generic materials, with specificity provided by pathogen-specific primers. In some other embodiments, a diagnostic detection tool or device can be purchased commercially. A typical detection tool or device usually includes an absorbent pad (a reservoir where the sample is placed), a paper or nitrocellulose membrane that serves as a support for wicking/movement of the mobile phase containing amplified DNA and that is imprinted with at least two perpendicular reagent lines bound to the membrane. These lines include a line consisting of anti-fluorescein antibody (sample line) and a line consisting of biotin (control line).

Two pathogen-specific primers can be used for the amplification step, one labeled with fluorescein and the other labeled with biotin. When DNA is amplified, a double-stranded amplicon is formed, labeled at one end with fluorescein and at the other with biotin. Also mobilized by wicking of the amplicon-containing sample, are streptavidin-labeled gold nanoparticles. As it moves across the sample line, the fluorescein-labeled end of the amplicon binds to the anti-fluorescein antibody on the membrane, immobilizing the amplicon. The nanoparticles bind to the biotin-labeled end of the amplicon, their aggregation at this line forms a visible red color, indicating presence of the amplicon. Additional nanoparticles bind to the biotin that is immobilized at the control line, providing a visual indicator that the assay is working properly, so that absence of a signal at the sample line represents absence of target and not a faulty assay.

In other embodiments, other approaches that involve enzymatic labels, such as horseradish peroxidase, can signal the presence of an amplicon through generation of colorimetric or fluorescent (more sensitive) signals.

In some embodiments, the magnetic ionic liquid used in the methods or kits disclosed herein comprises a paramagnetic anionic ligand and a paramagnetic cationic ligand.

In another aspect, disclosed herein is a kit for extracting, concentrating, detecting viable microbes from a sample, the kit comprises a magnetic ionic liquid as disclosed herein: an enzyme for Recombinase Polymerase Amplification (RPA); and a power-free heat source.

In another aspect, disclosed herein is a kit for extracting, concentrating, detecting viable microbes from a sample, the kit comprises a magnetic ionic liquid as disclosed herein; an enzyme for Recombinase Polymerase Amplification (RPA); and an extracting medium; wherein the extracting medium comprises more than 10 g/L of tryptone, 10 g/L of NaCl, 5 g/L of yeast extract, or combination thereof.

In yet another aspect, disclosed herein is a kit for extracting, concentrating, detecting viable microbes from a sample, the kit comprises a magnetic ionic liquid as disclosed herein; an enzyme for Recombinase Polymerase Amplification (RPA); and a primer for RPA, wherein the primer is one for Salmonella-specific DLH gene.

In some embodiments, wherein the extracting medium is a Luria-Bertani-derived nutrient broth comprising more than 10 g/L of tryptone, more than 5 g/L of yeast extract, more than 10 g/L of NaCl, or combination thereof. In some other embodiments, the extracting medium is a Luria-Bertani-derived nutrient broth comprising from more than 10 g/L to about 20 g/L of NaCl, from more than 10 g/L g/L to about 20 g/L of tryptone, from more than 5 g/L to about 20 g/L of yeast extract, or combination thereof.

In some embodiments, the kit further comprises an extracting medium. In some other embodiments, the extracting medium enhances the microbes' or their diagnostically useful cellular components', including, but not limited to DNA, RNA, other nucleic acids, proteins, enzymes, lipids or cell wall materials characteristic of the target organism, recovery, compared to using water. In some embodiments, the extracting medium is a Luria-Bertani-derived nutrient broth. In some other embodiments, the extracting medium comprises tryptone, yeast extract, NaCl, or combination thereof. In yet some other embodiments, the extracting medium is a Luria-Bertani-derived nutrient broth comprising tryptone, yeast extract, NaCl, or combination thereof.

In some embodiments, the kit further comprises a primer or primer set for RPA specifically designed for the microbes. In some other embodiments, the kit further comprises a primer or primer set for RPA specifically designed for Salmonella. In yet some other embodiments, the kit further comprises a DLH primer or primer set for RPA specifically designed for Salmonella. In some embodiments, the primer set is (Forward primer) 5′-GCC GGG CAG CRA TTA TTC TGC ATG AA-3′ and (Reverse primer) 5′-TGG CGT ATA CGG GAA CCG TAA TAG CA-3′.

In some embodiments, the kit further comprises a reverse transcriptase (RT). The RT enzyme generates a complementary DNA (cDNA) from the single-stranded rRNA template and the RPA enzyme system can use this cDNA as a substrate for DNA amplification.

In some embodiments, the kit further comprises a power-free heat source. In some other embodiments, the kit further comprises a chemical heat pack for RPA. A chemical heat pack is based on exothermic chemical reactions and may be regenerable. In some other embodiments, the kit further comprises a portable heat source for RPA powered by a battery or solar energy or light.

In some embodiments, the kit further comprises an amplicon detection tool or device. In some embodiments, the amplicon detection tool is a nucleic acid lateral flow immunoassay (NALFIA) disposable cartridge.

As used herein, a chemical heat source refers to a device that generate heat solely from the chemicals within the device, without using or transforming any other energy source, such as mechanical or electrical source into heat. A chemical heat source includes, but is not limited to, supercooled, food-grade sodium acetate pack.

As used herein, a portable heat source is a power-free heat source and includes a battery-powered heat source or a solar-powered heat source. As used herein, a battery-powered heat source refers to a device that convert the energy stored in a battery into heat. A battery-powered heat source includes, but is not limited to, a USB-powered incubator or battery powered infrared lamp.

As used herein, a solar-powered heat source refers to a device that convert the solar energy or natural light into heat.

As used herein, a power tool or a power equipment refers to a device that consumes electricity for performing its intended function.

As disclosed herein, a method of using a class of magnetic ionic liquids (MILs), which have very low water solubility, tunable chemical structure, low viscosity, suitable hydrophobicity and greater magnetic susceptibility, and RPA for determining microbes' identity and concentration is described. A kit for carrying out the disclosed method is also described.

Using the disclosed MILs for extraction or preconcentration of microbes can speed up the detection, identification, or quantification of the microbes, because the MILs can preconcentrate the microbes or eliminate other factors that might interfere or prevent the detection of the microbes. By dispersing hydrophobic MILs in an aqueous sample comprising microbes, the microbes can be rapidly extracted and isolated using an applied magnetic field. The extracted microbes were recovered from the MIL extraction phase by agitation in an extracting medium and subsequently the characteristic DNA of the microbes is amplified by RPA in a constant temperature.

Interestingly, the enrichment of viable Salmonella bacteria by MILs was dependent upon the identity of the paramagnetic metal incorporated into the chemical structure of the MIL, providing a basis for the design of MILs to exhibit enhanced cell extraction performance. Under optimized conditions, the MIL comprised of a trihexyl(tetradecyl)phosphonium cation ([P₆₆₆₁⁺]) and Ni(II) hexafluoroacetylacetonate-based anion ([Ni(hfacac)₃⁻]) was capable of enriching sufficient viable cells for the detection of viable Salmonella bacteria concentrations as low as about 10⁴ CFUs mL⁻¹ in a food sample with an extraction/recovery procedure of less than 10 min. The MIL-based extraction method was also coupled with RPA amplification for the rapid analysis of viable Salmonella bacteria, demonstrating the compatibility of MILs with both culture-based and nucleic acid-based methodologies for viable Salmonella bacteria detection.

Extracting or concentrating microbes from a sample and then using RPA to amplify microbes' characteristic DNA is one of the approaches to improve the existing microbe testing throughput, since doing so decreases the amount of time for proper identification and quantification. Furthermore, the methods or kits disclosed herein make microbes' detection and identification on site or in field possible, without the need for any power equipment or tool.

All publications, patent applications, issued patents, and other documents referred to in this specification are indicative of the level of ordinary skill in the art to which this disclosure pertains and are herein incorporated by reference to the same extent as if each individual publication or patent application was specifically and individually indicated as incorporated by reference. Definitions that are contained in text incorporated by reference are excluded to the extent that they contradict definitions in this disclosure.

### EXAMPLES

Embodiments of the enhancers disclosed herein and their methods of use are further defined in the following non-limiting Examples. It should be understood that these Examples, while indicating one or more preferred embodiments, are given by way of illustration only and are non-limiting. From the above discussion and these Examples, one skilled in the art can ascertain the essential characteristics of the disclosed compositions, and without departing from the spirit and scope thereof, can make various changes and modifications of the embodiments disclosed herein to adapt it to various usages and conditions. Thus, various modifications of the embodiments, in addition to those shown and described herein, will be apparent to those skilled in the art from the foregoing description. Such modifications are also intended to fall within the scope of the appended claims.

### Materials and Methods—Example 1

### Preparation and Characterization of Cationic DGE Ligands

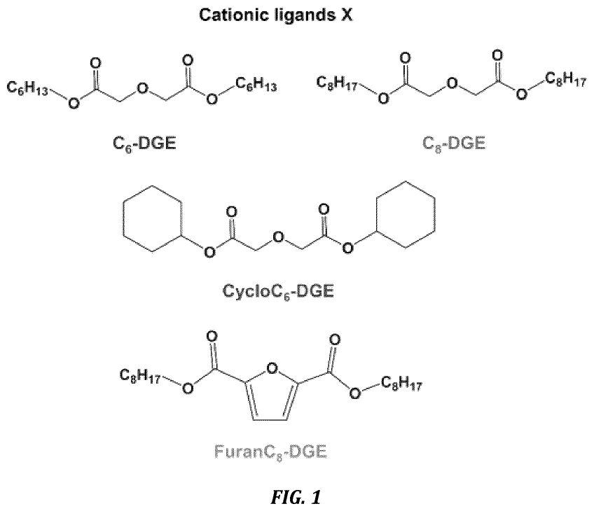

The structures of DGE ligands are shown in FIG. 1. A mixture of triethylamine (90 mmol) and 40 mmol of alcohol (1-hexanol, 1-octanol, or cyclohexanol) was placed in a 250 mL round bottom flask (RBF) and dissolved in 100 mL of dichloromethane. Diglycolyl chloride (2,2′-oxydiacetyl chloride, 20 mmol) was added dropwise to the contents of the flask and stirred for 6 hours under ambient conditions. The resulting solution was transferred to a 250 mL separatory funnel and washed 6 times with 30 mL of 10% HCl solution followed by a final wash with 50 mL of 5% (w/w) NaHCO₃ solution. Dichloromethane was removed under reduced pressure and the products were dissolved in 50 mL of hexane and filtered under gravity to remove any remaining salt impurities.

DGE ligands containing an aromatic substituent were prepared as follows. 2,5-furandicarboxylic acid (20 mmol) was transferred into a 250 mL RBF along with an excess (200 mmol) of thionyl chloride (SOCl₂). Five drops of N,N-dimethylformamide (N,N-DMF) were added to the flask and its contents were then refluxed at 50° C. for 24 hours until a homogenous clear solution was obtained. Excess SOCl₂ was removed under reduced pressure and the resulting furan-2,5-dicarbonyl dichloride was reacted with 1-octanol using the same procedure outlined for diglycolyl chloride.

The DGE ligands were characterized using ¹³C and ¹H NMR. NMR spectroscopy was conducted using Bruker 400 and 600 MHz spectrometers.

### Synthesis and Characterization of MILs

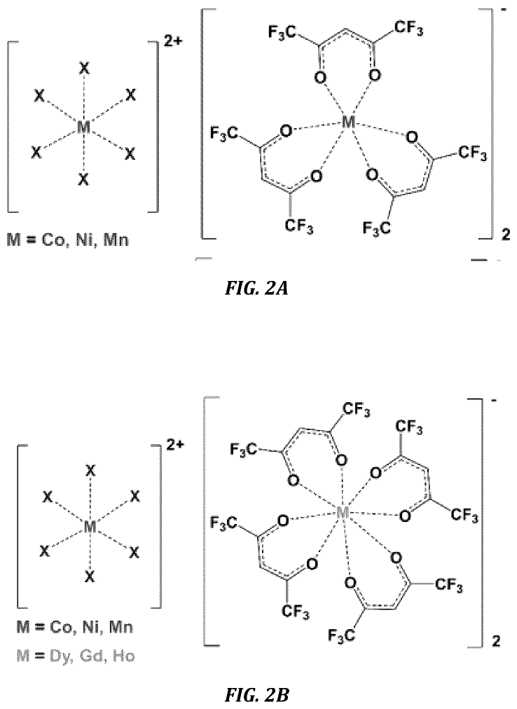

The chemical structure of MILs containing transition metals in the cation and anion are shown in FIG. 2A. The chemical structure of MILs containing transition and rare-earth metals in the cation and anion are shown in FIG. 2B.

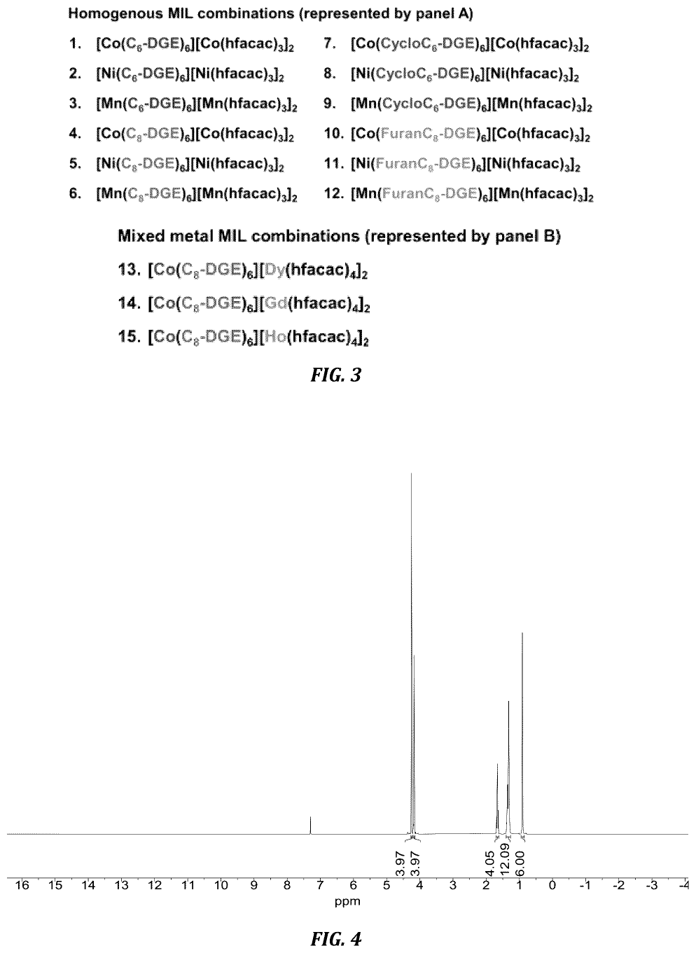

Homogenous and mixed metal MIL combinations are shown respectively in FIG. 3.

Homogenous MIL combinations were synthesized by using a one-pot synthesis approach. DGE ligands (5 mmol) were solubilized in 60 mL of methanol and reacted over 24 hours with 2.5 mmol of transition metal chlorides (NiCl₂, CoCl₂, and MnCl₂·4H₂O) in a 2:1 molar ratio to form chloride-based salts of the cation system. The resulting cationic salt was mixed with 5 mmol of [hfacac⁻] metal chelates composed of transition metal centers at a 1:2 molar ratio. Methanol was removed under reduced pressure and the contents of the flask were solubilized in 100 mL of diethyl ether and filtered under gravity to remove solid by-products. The filtrate was transferred to a 250 mL separatory funnel and repeatedly washed with 10 ml of water until the aqueous layer produced no precipitate through an anion-exchange mechanism with AgNO₃. The solvent was evaporated under reduced pressure and the MIL was dissolved in 60 mL of hexane. Any solid by-products and/or excess ammonium-based anion salts were removed by filtering under gravity and hexane was subsequently removed using rotary evaporation. The final MIL was dried under vacuum for 48 hours prior to further characterization.

To prepare mixed metal MIL combinations, the cobalt-containing cation salts were mixed with 5 mmol of [hfacac⁻] metal chelates of lanthanides (Dy, Gd, Ho) in the same molar ratio (1:2) and the purification steps were performed in a manner identical to that of transition metal magnetic ionic liquids.

Elemental analysis was performed using a Thermo Scientific FlashSmart 2000 CHNS/O Combustion Elemental Analyzer (Waltham, MA, USA).

### Effect of Cationic Ligands and Metal Centers of DGE-Based MILs on Viscosity

A Brookfield DVI cone and plate viscometer equipped with a CPA-51Z cone spindle was employed to obtain viscosity measurements at 21.6° C. using approximately 0.5 g of each MIL after extensive drying for 48 hours under vacuum. Viscosity measurements were conducted at 21.7° C. for 15 lanthanide and transition metal MILs examined in this study.

### Magnetic Properties of Transition-Based and Mixed Metal MILs

Magnetic properties were analyzed using a magnetic susceptibility balance (MSB, Sherwood Scientific, Cambridge, UK) and a Quantum Design Superconducting Quantum Interference Device (SQUID) magnetometer (MPMS XL-7). The magnetic properties of transition metal MILs were further studied using a SQUID magnetometer to confirm their paramagnetic nature as well as if any magnetic transition occurs in the samples.

Miscibility and Solvent Compatibility of DGE-based MILs with Volatile Organic Solvents

The solubility of lanthanide and transition metal MILs was investigated in popular traditional organic solvents. A 10 mg mass of each MIL was vigorously mixed with 100 μL of the desired solvent in a 1 mL glass vial to prepare MIL solutions at a concentration of 10% (w/v) MIL-to-solvent ratio.

### Thermal Stability of MILs Containing Multiple Paramagnetic Metal Centers

The thermal stability of MILs was measured from 40 to 350° C. at a ramp rate of 5° C. min⁻¹ by performing thermogravimetric analysis (TGA) using a NETZSCH STA 449 F1 Jupiter thermal analyzer (Selb, Germany). To further investigate the thermal stability of MILs using a more sensitive technique, the solvents were employed as a gas chromatographic (GC) stationary phase and subjected to an oven temperature program from 40 to 200° C. at a ramp rate of 1° C. min⁻¹ and decomposition/degradation products detected using an ultra-sensitive flame ionization detector (FID), with all measurements being performed on an Agilent Technologies 7890B gas chromatograph (Santa Clara, CA, USA).

### Magnetic Susceptibilities and Calculations for the Effective Magnetic Moment Using Magnetic Susceptibility Balance

Magnetic susceptibility (X_{M}) was calculated through Equation (1). In Equation (1), C_{bal} represents the balance calibration constant, L the length of MIL in the sample tube, M the molecular weight of the solvent, R the instrument reading displayed for the MIL in the sample tube, R₀ the initial reading for the empty tube, and m the mass of the MIL sample.

$${X}_{M} = [ {C}_{bal} LM ( R - {R}_{0} ) ] / ( {10}^{9} m ) \qquad ( 1 )$$

Effective magnetic moment (μ_{eff}) was calculated through Equation (2). In Equation (2), T represents the temperature at which the magnetic susceptibility measurements were conducted.

$${μ}_{eff} = 2.83 {( {X}_{M} T )}^{0.5} \qquad ( 2 )$$

Comparison of effective magnetic moment with theoretical effective paramagnetic moment (μₜₕₑₒᵣ) was conducted through Equation (3). In Equation (3), the μ_{X} refers to the theoretical effective magnetic moment for each metal center.

$${μ}_{theo r}^{2} = μ {x}^{2} + μ {x}^{2} + μ {x}^{2} \qquad ( 3 )$$

### Flow Analysis of Magnetic Ionic Liquids Under Gravity

The flows of magnetic ionic liquids were observed through inversion on glass slides. Snapshots were taken either 30 or 60 seconds after inversion.

### Thermogravimetric Analysis for Lanthanide and Transition Metal-Based Magnetic Ionic Liquids

Volitization/degradation of MILs as a function of temperature were measured using thermogravimetric analysis. The thermal stability of magnetic ionic liquids was measured from 40 to 350° C. at a ramp rate of 5° C. min⁻¹ by performing thermogravimetric analysis (TGA) using a NETZSCH STA 449 F1 Jupiter thermal analyzer (Selb, Germany).

### Example 1

### Ultra-Low Viscosity and High Magnetic Susceptibility Magnetic Ionic Liquids Featuring Functionalized Diglycolic Acid Ester Rare-Earth and Transition Metal Chelates

In this Example, diglycolic acid esters were employed as cationic ligands in the synthesis of fifteen magnetic ionic ligands comprised of multiple paramagnetic centers. Their physico-chemical properties were thoroughly characterized and benchmarked against previous classes of MILs. This study explores the distinct design of DGE-based MILs and demonstrates that such combinations produce compounds that are less viscous compared to many organic solvents, making them highly appealing in catalysis, organic synthesis, as well as chemical and electrochemical separations as a sustainable replacement for traditional solvents, where their paramagnetic properties can be readily exploited.

### Synthesis and Characterization of DGE Ligands

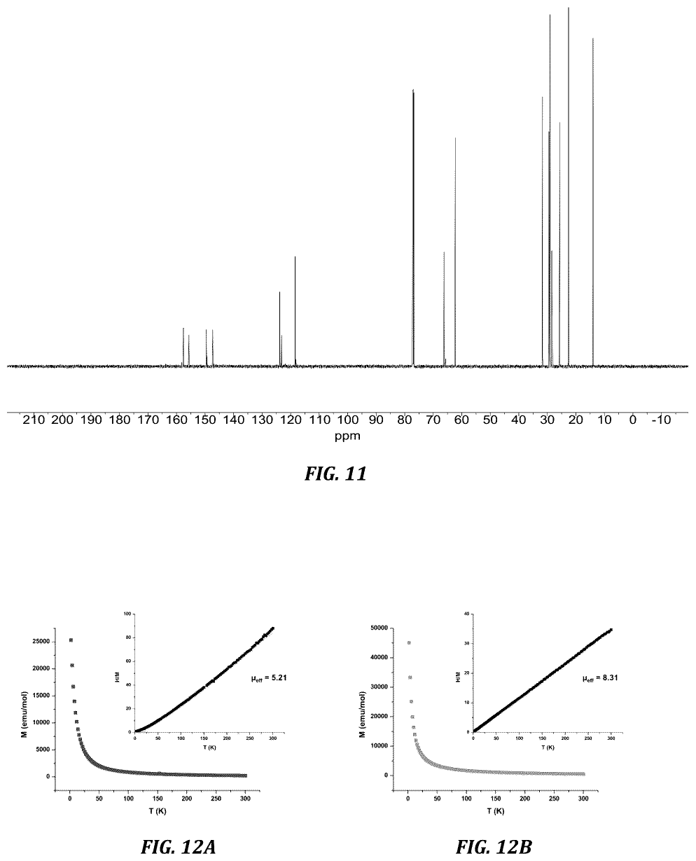

FIG. 1 shows the chemical structure of synthesized DGE ligands. NMR spectroscopy was conducted using Bruker 400 and 600 MHz spectrometers, and FIG. 4 (¹H NMR of dihexyl-2,2′-oxidiacetate (C₆-DGE) ligand) through FIG. 11 show the results of ¹H and ¹³C NMR upon the DGE ligand samples. FIG. 4 shows an ¹H NMR (600 MHZ, CDCl₃) δ 4.25 (s, 1H), 4.18 (t, J=6.8 Hz, 1H), 1.66 (p, J=6.9 Hz, 1H), 1.40-1.26 (m, 3H), 0.91 (t, J=6.8 Hz, 2H); pale yellow liquid; and yield of 97%. The NMR results are summarized in greater detail in the following paragraphs.

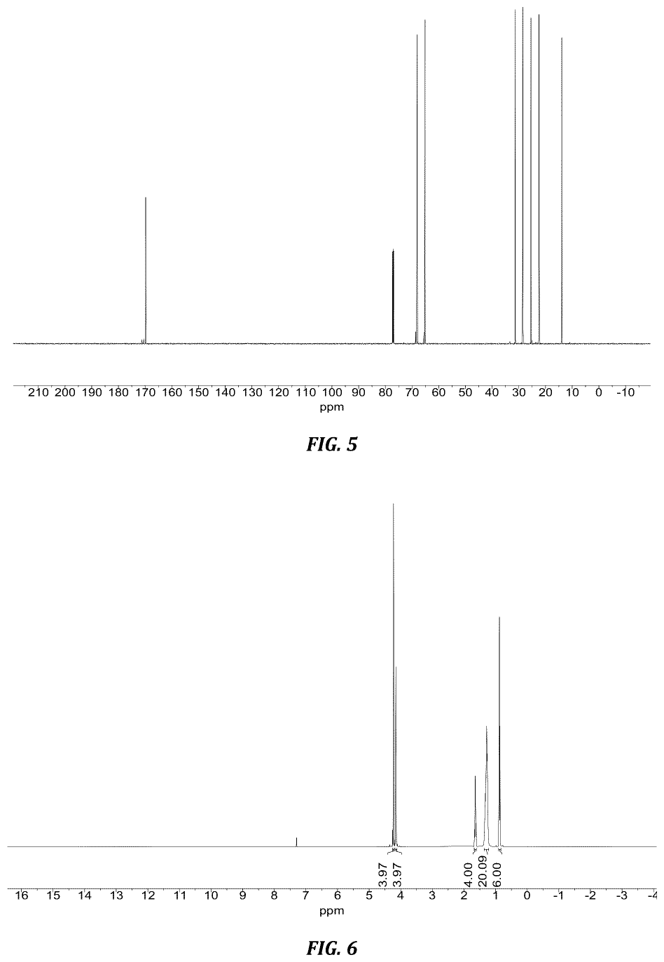

FIG. 5 is a graph from the ¹³C NMR testing of dihexyl-2,2′-oxidiacetate (C₆-DGE) ligand (151 MHZ, CDCl₃) providing δ 169.79, 68.65, 68.08, 65.55, 65.12, 31.33, 28.46, 28.42, 25.45, 22.45, 13.91; pale yellow liquid; and yield of 97%.

FIG. 6 is a graph from the ¹H NMR testing of dioctyl-2,2′-oxidiacetate (C₈-DGE) ligand (600 MHZ, CDCl₃) providing δ 4.23 (s, 4H), 4.15 (t, J=6.8 Hz, 4H), 1.68-1.61 (m, 4H), 1.36-1.22 (m, 20H), 0.88 (t, J=7.0 Hz, 6H); pale yellow liquid; and yield of 98%.

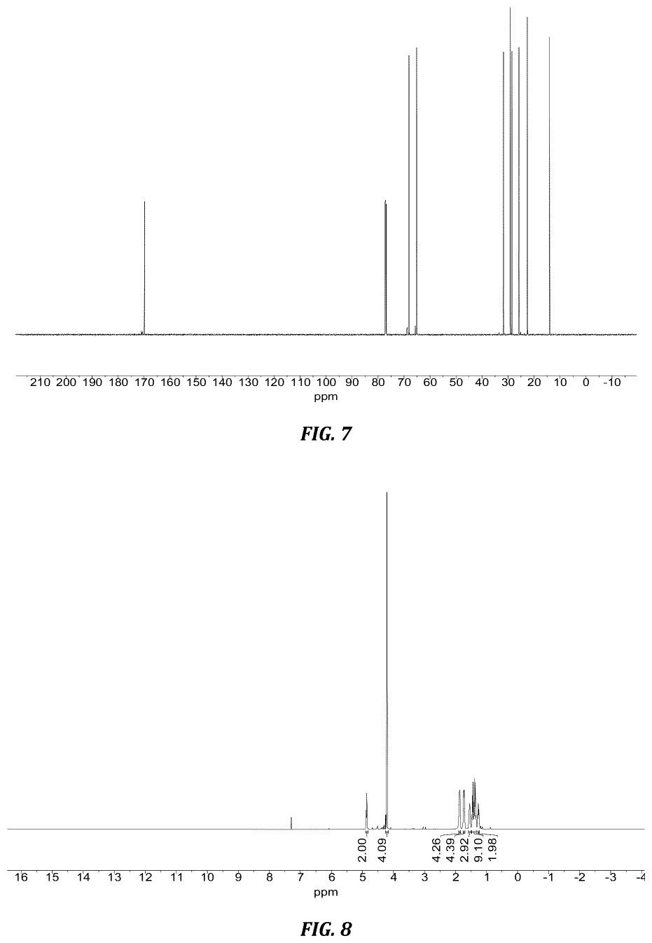

FIG. 7 is a graph from the ¹³C NMR testing of dioctyl-2,2′-oxidiacetate (C₈-DGE) ligand (151 MHZ, CDCl₃) providing δ 169.82, 68.11, 65.16, 31.74, 29.14, 29.12, 28.52, 28.47, 25.80, 25.77, 22.60, 14.04; pale yellow liquid; and yield of 98%.

FIG. 8 is a graph from the ¹H NMR (600 MHZ, CDCl₃) testing of dicyclohexyl-2,2′-oxidiacetate (CycloC₆-DGE) providing 8 4.86 (ddp, J=13.4, 8.5, 4.6 Hz, 2H), 4.21 (s, 4H), 1.90-1.84 (m, 4H), 1.76-1.70 (m, 4H), 1.59-1.50 (m, 3H), 1.48-1.33 (m, 9H), 1.28-1.23 (m, 2H); yellow liquid; and yield of 97%.

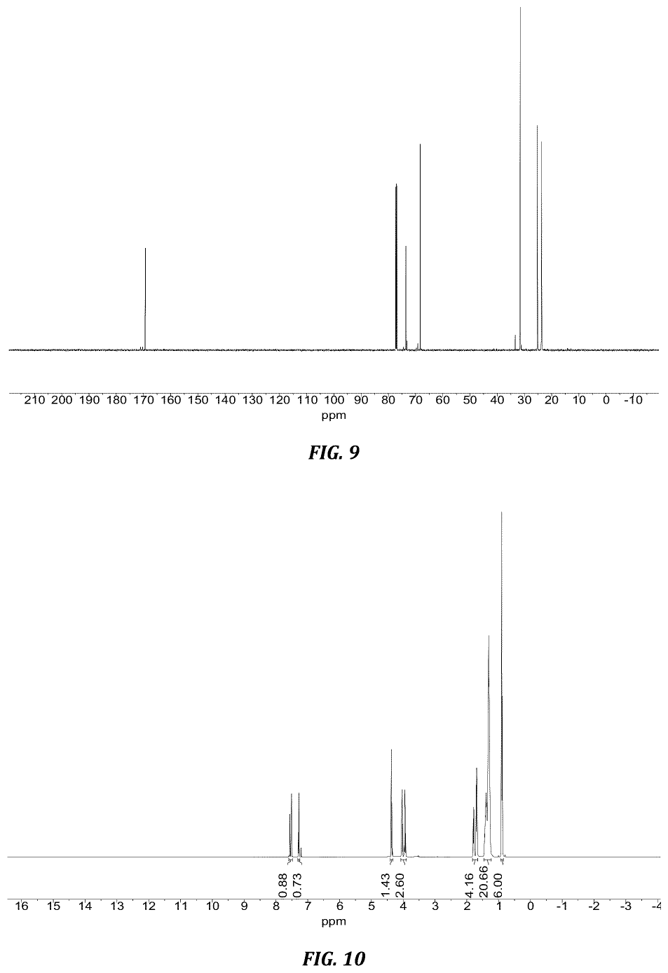

FIG. 9 is a graph from the ¹³C NMR of dicyclohexyl-2,2′-oxidiacetate (CycloC₆-DGE) (151 MHz, CDCl₃) providing δ 169.26, 73.57, 73.19, 68.32, 33.45, 33.38, 31.55, 31.50, 25.26, 25.14, 23.79, 23.68.; yellow liquid; and yield of 97%.

FIG. 10 is a graph from the ¹H NMR of dioctylfuran-2,5-dicarboxylate (FuranC₈-DGE) ligand (600 MHZ, CDCl₃) providing δ 7.60-7.48 (m, 1H), 7.29 (d, J=7.4 Hz, 1H), 4.37 (t, J=6.8 Hz, 1H), 3.99 (ddt, J=55.3, 10.1, 6.6 Hz, 3H), 1.74 (dp, J=56.3, 6.9 Hz, 4H), 1.46-1.23 (m, 21H), 0.90 (t, J=6.9 Hz, 6H); dark yellow liquid that turns into an amorphous solid over time; and yield of 95%.

FIG. 11 is a graph from the ¹³C NMR of dioctylfuran-2,5-dicarboxylate (FuranC₈-DGE) ligand (151 MHz, CDCl₃) providing δ 157.61, 155.84, 155.67, 149.59, 147.34, 123.87, 123.20, 118.45, 66.27, 62.33, 31.76, 29.52, 29.15, 29.14, 29.12, 28.52, 25.81, 25.77, 22.63, 14.07. Dark yellow liquid that turns into an amorphous solid over time. Yield of 95%.

### Synthesis and Optimization of Lanthanide and Transition Metal MILs

To optimize the synthetic route, it was important to determine the number of DGE ligands required to form a single cation unit. Given that transition metals were previously found to chelate with DGA ligands in a 1:2 stoichiometry to form the cation, the same molar ratio was employed between metal chlorides and DGE ligands. As all reagents and intermediates were completely soluble in methanol, a one-pot synthesis method was preferred to prepare the chloride-based cationic salt followed by an anion exchange reaction with the ammonium-based [hfacac⁻] metal salts of both lanthanides and transition metals in a 1:2 molar ratio to produce the final MIL. CHN elemental analysis displayed that the transition metal center was coordinated to six DGE ligands, and only 3 hexafluoroacetylacetonate ligands were chelated to the transition metals compared to lanthanides. Based on these molar ratios, DGE ligands were identified as the limiting reagent and hence, excess metal chloride used in synthesis of the cation system was removed by the aqueous layer during the washing step. Only the final MIL was soluble in hexane and any excess reagent/intermediate was removed as a precipitate.

Homogenous MIL combinations, shown in FIG. 1 as MIL 1 to 12, were synthesized using the aforementioned one-pot synthetic approach. The MIL product was purified and dried under vacuum for 48 hours.

Mixed metal MIL combinations, shown in FIG. 1 as MIL 13 to 15, were synthesized using the aforementioned one-pot synthetic approach. The MIL product was purified and dried under vacuum for 48 hours.

CHN elemental analysis was conducted upon the MIL products using a Thermo Scientific FlashSmart 2000 CHNS/O Combustion Elemental Analyzer (Waltham, MA, USA). This analysis provided for the characterization of purities of the synthesized MILs.
a. MIL 1: Dark red, non-viscous liquid. Elemental analysis calculated (%) for Co₃C₁₂₆H₁₈₆F₃₆O₄₂: C, 46.80; H, 5.80; N, 0.00. Found: C, 46.33; H, 5.98; N, 0.14.
b. MIL 2: Dark green, non-viscous liquid. Elemental analysis calculated (%) for Ni₃C₁₂₆H₁₈₆F₃₆O₄₂: C, 46.81; H, 5.80; N, 0.00. Found: C, 46.88; H, 6.38; N, 0.41.
c. MIL 3: Dark yellow, non-viscous liquid. Elemental analysis calculated (%) for Mn₃C₁₂₆H₁₈₆F₃₆O₄₂: C, 46.98; H, 5.82; N, 0.00. Found: C, 47.96; H, 5.26; N, 0.04.
d. MIL 4: Bright red, non-viscous liquid. Elemental analysis calculated (%) for Co₃C₁₅₀H₂₃₄F₃₆O₄₂: C, 50.46; H, 6.61; N, 0.00. Found: C, 51, 12; H, 6.33; N, 0.57.
e. MIL 5: Bright green, non-viscous liquid. Elemental analysis calculated (%) for Ni₃C₁₅₀H₂₃₄F₃₆O₄₂: C, 50.47; H, 6.61; N, 0.00. Found: C, 50.48; H, 7.41; N, 0.50.
f. MIL 6: Bright yellow, non-viscous liquid. Elemental analysis calculated (%) for Mn₃C₁₅₀H₂₃₄F₃₆O₄₂: C, 50.63; H, 6.63; N, 0.00. Found: C, 50.13; H, 7.02; N, 0.15.
g. MIL 7: Red, non-viscous liquid. Elemental analysis calculated (%) for Co₃C₁₂₆H₁₆₂F₃₆O₄₂: C, 47.16; H, 5.09; N, 0.00. Found: C, 45.27; H, 5.49; N, 0.200.
h. MIL 8: Green, non-viscous liquid. Elemental analysis calculated (%) for Ni₃C₁₂₆H₁₆₂F₃₆O₄₂: C, 47.17; H, 5.09; N, 0.00. Found: C, 47.11; H, 5.83; N, 0.14.
i. MIL 9: Yellow, non-viscous liquid. Elemental analysis calculated (%) for Mn₃C₁₂₆H₁₆₂F₃₆O₄₂: C, 47.33; H, 5.11; N, 0.00. Found: C, 47.91; H, 5.98; N, 0.13.
j. MIL 10: Bright red, non-viscous liquid that becomes semi-solid over time. Elemental analysis calculated (%) for Co₃C₁₆₂H₂₂₂F₃₆O₄₂: C, 52.56; H, 6.04; N, 0.00. Found: C, 52.70; H, 7.42; N, 0.24.
k. MIL 11: Bright green, non-viscous liquid that becomes semi-solid over time. Elemental analysis calculated (%) for Ni₃C₁₆₂H₂₂₂F₃₆O₄₂: C, 52.57; H, 6.05; N, 00. Found: 52.78; H, 7.52; N, 0.50.
l. MIL 12: Bright yellow, non-viscous liquid that becomes semi-solid over time. Elemental analysis calculated (%) for Mn₃C₁₆₂H₂₂₂F₃₆O₄₂: C, 52.73; H, 6.06; N, 0.00. Found: C, 49.03; H, 6.25; N, 0.23.
m. MIL 13: Pale red, non-viscous liquid. Elemental analysis calculated (%) for CoDy₂C₁₆₀H₂₄₄F₄₈O₄₆: C, 45.76; H, 5.86; N, 0.00. Found: C, 47.84; H, 5.83; N, 0.19.
n. MIL 14: Pale red, non-viscous liquid. Elemental analysis calculated (%) for CoGd₂C₁₆₀H₂₄₄F₄₈O₄₆: C, 45.88; H, 5.87; N, 0.00. Found: C, 44.81; H, 5.99; N, 0.19.
o. MIL 15: Pale red, non-viscous liquid. Elemental analysis calculated (%) for CoH₀₂C₁₆₀H₂₄₄F₄₈O₄₆: C, 45.71; H, 5.85; N, 0.00. Found: C, 45.12; H, 4.78; N, 0.17.

### Effect of Cationic Ligands and Metal Centers of DGE-Based MILs on Viscosity

Viscosity measurements were conducted at 21.7° C. for 15 lanthanide and transition metal MILs examined in this study. TABLE 1, which displays the synthesized MILs with their respective viscosities, theoretical effective paramagnetic moments, and effective magnetic moments, shows that all MILs exhibited viscosities lower than 827.6 cP under ambient conditions with the lowest value of 31.6 cP obtained for the [Co(C₈-DGE)₆][Co(hfacac)₃]₂ MIL.

TABLE 1

| MIL No. | MIL abbreviation | Viscosity (cP) | μ [symbol] (μ [symbol] ) | μ_{eff} (μ [symbol] )^{a} |
|---|---|---|---|---|
| 1 | [Co(C₆-DGE)₆][Co(hfacac)₃]₂ | 175.0 | 8.30 | 8.11 |
| 2 | [Ni(C₆-DGE)₆][Ni(hfacac)₃]₂ | 248.6 | 5.37 | 5.48 |
| 3 | [Mn(C₆-DGE)₆][Mn(hfacac)₃]₂ | 63.2 | 10.13 | 10.42 |
| 4 | [Co(C₈-DGE)₆][Co(hfacac)₃]₂ | 31.6 | 8.30 | 8.57 |
| 5 | [Ni(C₈-DGE)₆][Ni(hfacac)₃]₂ | 119.1 | 5.37 | 5.33 |
| 6 | [Mn(C₈-DGE)₆][Mn(hfacac)₃]₂ | 96.3 | 10.13 | 10.25 |
| 7 | [Co(CycloC₆-DGE)₆][Co(hfacac)₃]₂ | 639.4 | 8.30 | 8.11 |
| 8 | [Ni(CycloC₆-DGE)₆][Ni(hfacac)₃]₂ | 827.6 | 5.37 | 5.53 |
| 9 | [Mn(CycloC₆-DGE)₆][Mn(hfacac)₃]₂ | 478.9 | 10.13 | 9.96 |
| 10 | [Co(FuranC₈-DGE)₆][Co(hfacac)₃]₂ | 354.7^{b} | 8.30 | 8.27 |
| 11 | [Ni(FuranC₈-DGE)₆][Ni(hfacac)₃]₂ | 453.5^{b} | 5.37 | 5.51 |
| 12 | [Mn(FuranC₈-DGE)₆][Mn(hfacac)₃]₂ | 291.8^{b} | 10.13 | 10.04 |
| 13 | [Co(C₈-DGE)₆][Dy(hfacac)₄]₂ | 137.2 | 15.56 | 15.38 |
| 14 | [Co(C₈-DGE)₆][Gd(hfacac)₄]₂ | 149.1 | 12.17 | 12.43 |
| 15 | [Co(C₈-DGE)₆][Ho(hfacac)₄]₂ | 216.4 | 15.63 | 15.56 |

^{a}Effective magnetic moment calculated using magnetic susceptibility measurements performed on a magnetic susceptibility balance
^{b}MIL turns into an amorphous solid when left to stand overnight
^{c}Viscosity measurement reported at 23.7° C.
^{d} Viscosity measurement reported at 20.0° C.
^{e}Viscosity measurement reported at 19.9° C.
^{f}Effective magnetic moment calculated using magnetic susceptibility measurements performed on a SQUID magnetometer [symbol]
indicates data missing or illegible when filed

Compared to the [Co(C₆-DGE)₆][Co(hfacac)₃]₂ MIL (175.0 cP), a significantly higher value of 639.4 cP was observed for the [Co(CycloC₆-DGE)₆][Co(hfacac)₃]₂ MIL, demonstrating that MILs comprised of cyclic alkyl substituents offer stronger intermolecular forces. Similar observations were made for Ni and Mn-based MILs comprised of the same ligands by comparing MILs 2 and 3 with MILs 8 and 9, further exemplifying that structural isomers of cationic ligands can significantly influence MIL viscosity.

The lowest viscosities were generally observed for solvents comprised of the C₈-DGE ligand with values of 31.6, 119.1, and 96.3 cP being obtained for the Co, Ni, and Mn MILs (MILs 4 to 6), respectively. Installing aromatic moieties in the cationic ligands (FuranC₈-DGE) produced a moderately high viscosity Co MIL (354.7 cP) compared to MIL 6 prepared using the C₈-DGE ligand with identical metal centers in the cation/anion.

In general, the highest viscosity solvent using any DGE ligand was obtained for Ni-based MILs compared to those comprised of Co and Mn metal centers, which can be attributed to the smaller atomic radius of nickel leading to a compact cation and stronger intermolecular forces.

Despite forming larger anions, incorporating lanthanides in mixed metal combinations (MILs 13 to 15) did not dramatically lower the MIL viscosity compared to those comprised of transition metals in both the cation and anion. Irrespective of the type of metal center, the size of the cation coordinated to the DGE ligands is perceived to be larger than that of the DGA ligands due to a larger number of cationic ligands chelated to the metal.

TABLE 2 shows the individual viscosities of previously studied DGA ligands.

TABLE 2

| | | | |
|---|---|---|---|
| [P₆₆₆₁₄][Co(hfacac)₃] | 575.8^{c} | 4.75 | 4.30^{f} |
| [P₆₆₆₁₄][Ni(hfacac)₃] | 927.9^{c} | 3.10 | 2.80^{f} |
| [P₆₆₆₁₄][Mn(hfacac)₃] | 401.8^{c} | 5.85 | 5.80^{f} |
| [P₆₆₆₁₄]₃[GdCl₆] | 28,230^{d} | 7.95 | 7.86^{f} |
| [P₆₆₆₁₄]₂[CoCl₄] | 123,500^{d} | 4.75 | 4.45^{f} |
| [P₆₆₆₁₄]₂[MnCl₄] | 112,300^{d} | 5.85 | 5.81^{f} |
| [Co(TODGA)₂][Co(hfacac)₃]₂ | 28,310^{e} | 8.30 | 13.34^{f} |
| [Ni(TODGA)₂][Ni(hfacac)₃]₂ | 33,830^{e} | 5.37 | 11.79^{f} |
| [Dy(TODGA)₃][Dy(hfacac)₄]₃ | 9,210^{e} | 21.30 | 20.00^{f} |
| [Gd(TODGA)₃][Gd(hfacac)₄]₃ | 13,640^{e} | 15.90 | 14.90^{f} |
| [Ho(TODGA)₃][Ho(hfacac)₄]₃ | 10,530^{e} | 21.20 | 20.86^{f} |

^{a} Effective magnetic moment calculated using magnetic susceptibility measurements performed on a magnetic susceptibility balance
^{b} MIL turns into an amorphous solid when left to stand overnight
^{c}Viscosity measurement reported at 23.7° C.
^{d}Viscosity measurement reported at 20.0° C.
^{e}Viscosity measurement reported at 19.9° C.
^{f}Effective magnetic moment calculated using magnetic susceptibility measurements performed on a SQUID magnetometer

As shown in TABLE 1 and TABLE 2, neat DGE ligands' viscosity were observed to be significantly lower than the previously employed DGA ligands.

### Magnetic Properties of Transition-Based and Mixed Metal MILs

15 MILs incorporating rare-earth and transition metals were examined using a magnetic susceptibility balance (MSB).

As shown in TABLE 1, the experimentally determined μ_{eff} values for the MILs were found to range from 5.33 to 15.56μ_{B} per formula unit, which matches quite well with corresponding theoretically calculated values. Compared to a value of 5.48μ_{B} for the [Ni(C₆-DGE)₆][Ni(hfacac)₃]₂ MIL, the μ_{eff} was approximately double for the [Mn(C₆-DGE)₆][Mn(hfacac)₃]₂ MIL (10.42μ_{B}). Varying the cationic ligand did not alter the μ_{eff} as values of 8.57, 8.11, and 8.27μ_{B} were obtained for the [Co(C₈-DGE)₆][Co(hfacac)₃]₂, [Co(CycloC₆-DGE)₆][Co(hfacac)₃]₂, and [Co(FuranC₈-DGE)₆][Co(hfacac)₃]₂ MILs, demonstrating that magnetic susceptibility is primarily dependent on the type of paramagnetic metal center. Higher μ_{eff} values were observed for the [Co(C₈-DGE)₆][Gd(hfacac)₄]₂ (12.43μ_{B}) and [Co(C₈-DGE)₆][Hi(hfacac)₄]₂ (15.56μ_{B}) mixed metal MILs due to the presence of multiple lanthanides in the anion.

Irrespective of the type of cationic ligands, all Mn and Co MILs formed high spin complexes, given the precise match between the μₜₕₑₒ and μ_{eff} values calculated for high spin states. Additionally, the μ_{eff} for DGE-based MILs was found to be comparable to those formed using DGA ligands and approximately double that of those comprised of phosphonium cations.

The magnetic properties of transition metal MILs were further studied using a SQUID magnetometer to confirm their paramagnetic nature as well as if any magnetic transition occurs in the samples.

FIG. 12 shows the magnetization of Ni and Mn-based MILs plotted as a function of temperature. The calculated μ_{eff} of 5.21μ_{B} for the [Ni(C₆-DGE)₆][Ni(hfacac)₃]₂ MIL (FIG. 12A) from SQUID matches quite well with the theoretical μₜₕₑₒ value (5.31μ_{B}, TABLE 1) and also with that obtained from MSB measurements (5.48μ_{B}). However, for the [Mn(C₆-DGE)₆][Mn(hfacac)₃]₂ MIL, the μ_{eff} value of 8.31μ_{B} obtained using SQUID appears to be significantly lower than theoretical value of 10.13μ_{B} (FIG. 12B, TABLE 1). More specifically, FIG. 12 shows the magnetization of Ni and Mn-based MILs studied as a function of temperature under an applied magnetic field of H=20 kOe: (A) [Ni(C₆-DGE)₆][Ni(hfacac)₃]₂ and (B) [Mn(C₆-DGE)₆][Mn(hfacac)₃]₂. The inset shows the Curie-Weiss fit of the linear portion of the reciprocal susceptibility.

Nevertheless, due to high sensitivity of the technique, magnetic susceptibility measurements using SQUID unambiguously prove that the prepared MIL samples exhibit paramagnetic behavior in the temperature range of 5-300 K without any signature of secondary magnetic phase as well as ordering.

Miscibility and Solvent Compatibility of DGE-Based MILs with Volatile Organic Solvents

The solubility of lanthanide and transition metal MILs was investigated in popular traditional organic solvents. TABLE 3, which displays the solubility of each MIL as well as the temperature of onset MIL volatilization, is shown for the solubility of each MIL in 14 polar and non-polar solvents at different concentrations, and further includes previously referenced MILs.

TABLE 3

| | | | Onset of MIL | Onset of MIL |
|---|---|---|---|---|
| | | | volatilization | volatilization |
| MIL No. | MIL abbreviation | Solubility | (° C.)¹ | (° C.)² |
| 1 | [Co(C₆-DGE)₆][Co(hfacac)₃]₂ | S^{a}S^{b}S^{c}S^{d}S^{e}S^{f} | 168 | 255.3 |
| 2 | [Ni(C₆-DGE)₆][Ni(hfacac)₃]₂ | S^{a}S^{b}S^{c}S^{d}S^{e}S^{f} | 166 | 257.0 |
| 3 | [Mn(C₆-DGE)₆][Mn(hfacac)₃]₂ | S^{a}S^{b}S^{c}S^{d}S^{e}S^{f} | 168 | 256.8 |
| 4 | [Co(C₈-DGE)₆][Co(hfacac)₃]₂ | S^{a}S^{b}S^{c}S^{d}S^{f} | 185 | 262.9 |
| 5 | [Ni(C₈-DGE)₆][Ni(hfacac)₃]₂ | S^{a}S^{b}S^{c}S^{d}S^{f} | 190 | 264.4 |
| 6 | [Mn(C₈-DGE)₆][Mn(hfacac)₃]₂ | S^{a}S^{b}S^{c}S^{d}S^{f} | 182 | 259.1 |
| 7 | [Co(CycloC₆-DGE)₆][Co(hfacac)₃]₂ | S^{a}S^{b}S^{c}S^{d}S^{e}S^{f} | 133 | 200.1 |
| 8 | [Ni(CycloC₆-DGE)₆][Ni(hfacac)₃]₂ | S^{a}S^{b}S^{c}S^{d}S^{e}S^{f} | 154 | 182.2 |
| 9 | [Mn(CycloC₆-DGE)₆][Mn(hfacac)₃]₂ | S^{a}S^{b}S^{c}S^{d}S^{e}S^{f} | 159 | 176.9 |
| 10 | [Co(FuranC₈-DGE)₆][Co(hfacac)₃]₂ | S^{a}S^{b}S^{c}S^{d}S^{f} | 147 | 163.0 |
| 11 | [Ni(FuranC₈-DGE)₆][Ni(hfacac)₃]₂ | S^{a}S^{b}S^{c}S^{d}S^{f} | 142 | 160.6 |
| 12 | [Mn(FuranC₈-DGE)₆][Mn(hfacac)₃]₂ | S^{a}S^{b}S^{c}S^{d}S^{f} | 144 | 160.4 |
| 13 | [Co(C₈-DGE)₆][Dy(hfacac)₄]₂ | S^{a}S^{b}S^{c}S^{d}S^{f} | 131 | 182.3 |
| 14 | [Co(C₈-DGE)₆][Gd(hfacac)₄]₂ | S^{a}S^{b}S^{c}S^{d}S^{f} | 126 | 172.9 |
| 15 | [Co(C₈-DGE)₆][Ho(hfacac)₄]₂ | S^{a}S^{b}S^{c}S^{d}S^{f} | 115 | 183.3 |
| — | [P₆₆₆₁₄][Co(hfacac)₃] | S^{a}S^{b}S^{f} | 130 | [symbol] |
| — | [P₆₆₆₁₄][Ni(hfacac)₃] | S^{a}S^{b}S^{f} | 225 | [symbol] |
| — | [P₆₆₆₁₄][Mn(hfacac)₃] | S^{a}S^{b}S^{f} | 155 | [symbol] |
| — | [Co(TODGA)₂][Co(hfacac)₃]₂ | S^{a}S^{b}S^{f} | 231 | [symbol] |
| — | [Ni(TODGA)₂][Ni(hfacac)₃]₂ | S^{a}S^{b}S^{f} | 235 | [symbol] |
| — | [Dy(TODGA)₃][Dy(hfacac)₄]₃ | S^{a}S^{b}S^{c}S^{f} | 175 | [symbol] |
| — | [Gd(TODGA)₃][Gd(hfacac)₄]₃ | S^{a}S^{b}S^{c}S^{f} | 183 | [symbol] |
| — | [Ho(TODGA)₃][Ho(hfacac)₄]₃ | S^{a}S^{b}S^{c}S^{f} | 192 | [symbol] |

¹MIL degradation temperature measured using GC-FID approach. Onset of MIL volatilization/degradation was determined by monitoring an exponential increase in response beyond a value of 2,000 pA of the detector.
²MIL degradation temperature measured using TGA at 10% weight loss
S^{a} = soluble in hexane, heptane, benzene, and toluene at 10% (w/v).
S^{b} = soluble in acetone, chloroform, dichloromethane, acetonitrile, methanol, ethanol, diethyl ether, ethyl acetate, and isopropanol at 20% (w/v).
S^{c} = soluble in hexane, heptane, benzene, and toluene at 20% (w/v).
S^{d} = soluble in acetone, chloroform, dichloromethane, methanol, ethanol, diethyl ether, ethyl acetate, isopropanol hexane, heptane, benzene, and toluene at 50% (w/v).
S^{e} = insoluble in acetonitrile at 50% (w/v).
S^{f} = insoluble in water at 0.01% (w/v). [symbol]
MIL characterization not reported using TGA [symbol]
indicates data missing or illegible when filed

All MILs were soluble water-immiscible solvents, such as benzene, up to concentrations of 50% (w/v) MIL-to-solvent ratio, demonstrating their hydrophobic character. Varying the type of metal center or ligand had no significant influence on the solubility of MILs in organic solvents, such as methanol and 2-propanol, at a concentration of 20% (w/v) MIL-to-solvent ratio.

However, MILs comprised of C₆-DGE and CycloC₆-DGE (MILs 1 to 3 and 7 to 9) were found to be insoluble in acetonitrile at a concentration of 50% (w/v) MIL-to-solvent ratio compared to those prepared using octyl-based ligands. Additionally, all MILs were insoluble in water at concentrations as low as 0.01% (w/v) MIL-to-solvent ratio.

### Thermal Stability of MILs Containing Multiple Paramagnetic Metal Centers

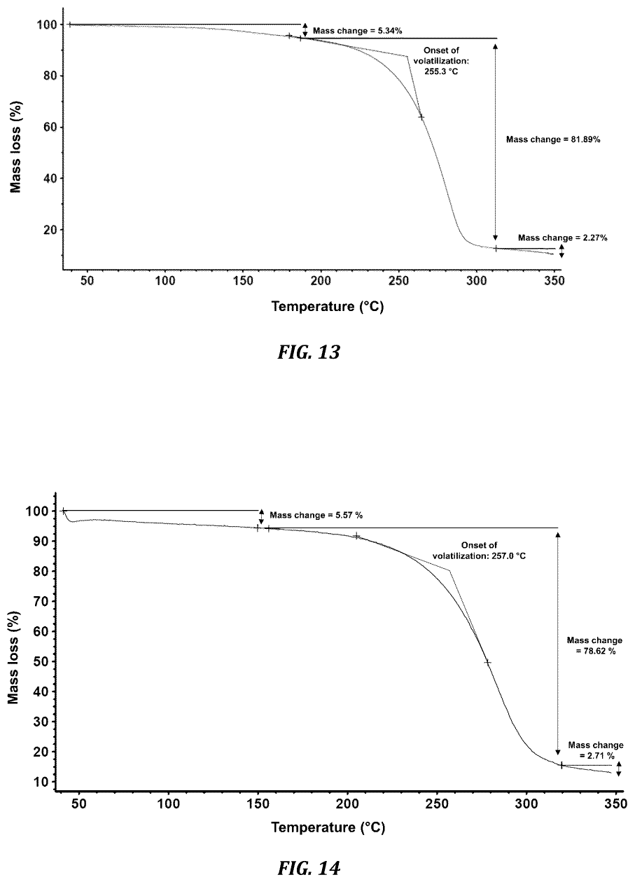

To investigate the ability of DGE-based MILs to withstand elevated temperatures without undergoing degradation/decomposition, their thermal stabilities were investigated using thermogravimetric analysis (TGA). FIG. 13 through FIG. 27 display thermograms for MILs 1 to 15. The thermogram results are summarized in greater detail in the following paragraphs.

FIG. 13 shows the volatilization/degradation of the [Co(C₆-DGE)₆][Co(hfacac)₃]₂ MIL as a function of temperature measured using TGA.

FIG. 14 shows the volatilization/degradation of the [Ni(C₆-DGE)₆][Ni(hfacac)₃]₂ MIL as a function of temperature measured using TGA.

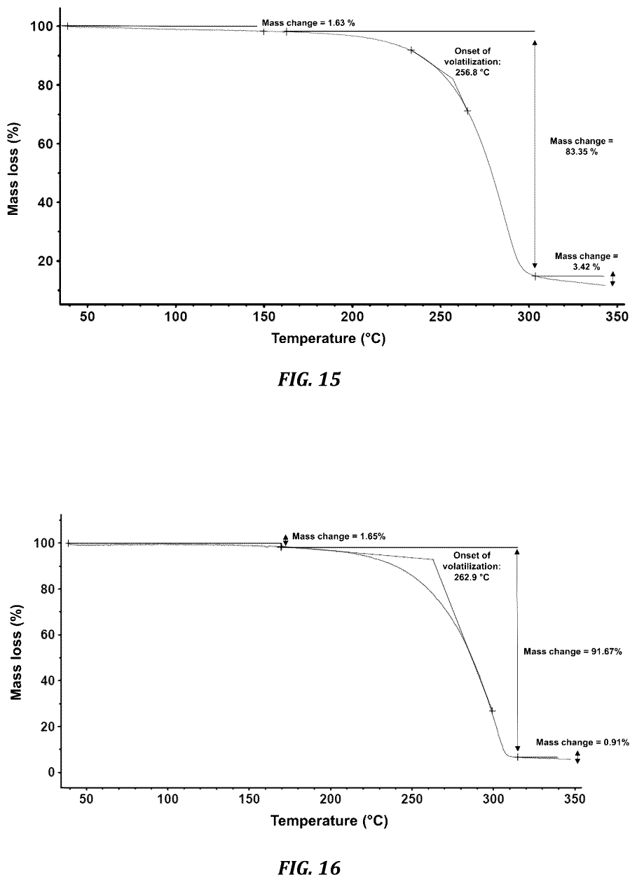

FIG. 15 shows the volatilization/degradation of the [Mn(C₆-DGE)₆][Mn(hfacac)₃]₂ MIL as a function of temperature measured using TGA.

FIG. 16 shows the volatilization/degradation of the [Co(C₈-DGE)₆][Co(hfacac)₃]₂ MIL as a function of temperature measured using TGA.

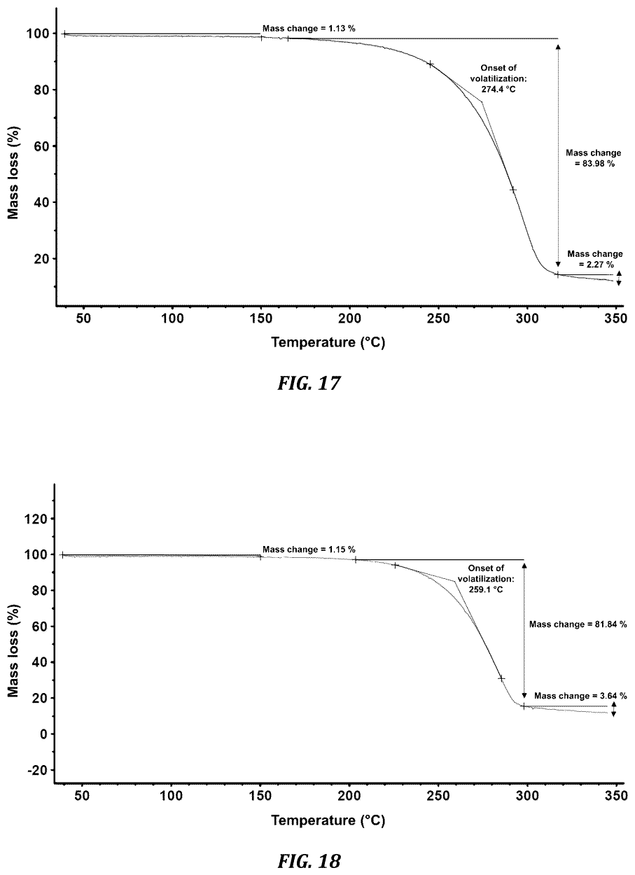

FIG. 17 shows the volatilization/degradation of the [Ni(C₈-DGE)₆][Ni(hfacac)₃]₂ MIL as a function of temperature measured using TGA.

FIG. 18 shows the volatilization/degradation of the [Mn(C₈-DGE)₆][Mn(hfacac)₃]₂ MIL as a function of temperature measured using TGA.

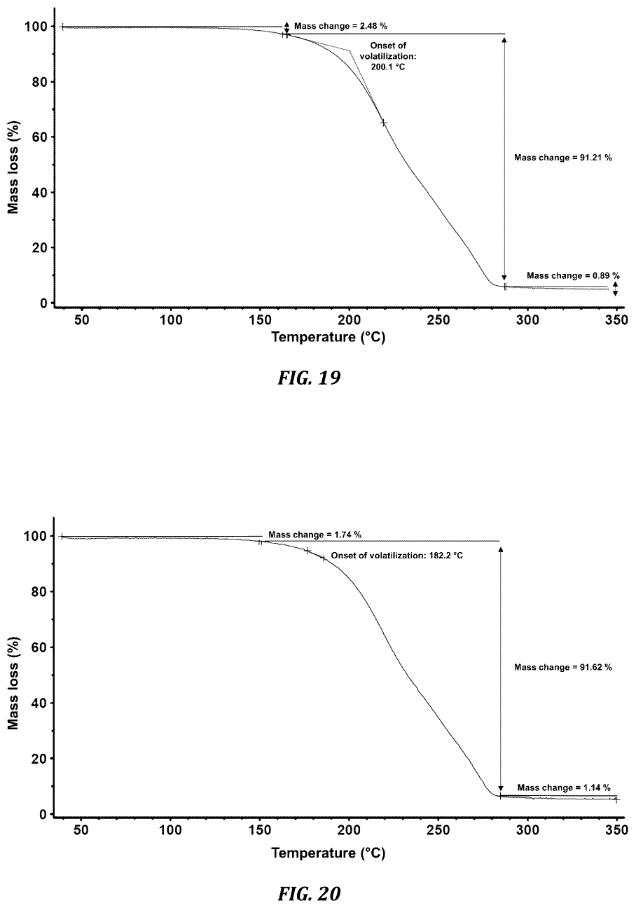

FIG. 19 shows the volatilization/degradation of the [Co(CycloC₆-DGE)₆][Co(hfacac)₃]₂ MIL as a function of temperature measured using TGA.

FIG. 20 shows the volatilization/degradation of the [Ni(CycloC₆-DGE)₆][Ni(hfacac)₃]₂ MIL as a function of temperature measured using TGA.

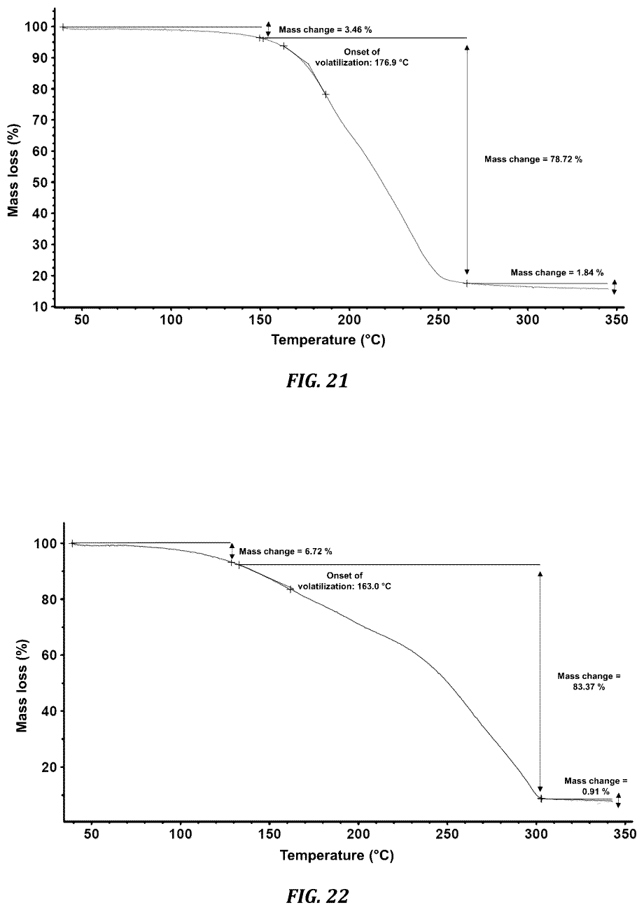

FIG. 21 shows the volatilization/degradation of the [Mn(CycloC₆-DGE)₆][Mn(hfacac)₃]₂ MIL as a function of temperature measured using TGA.

FIG. 22 shows the volatilization/degradation of the [Co(FuranC₈-DGE)₆][Co(hfacac)₃]₂ MIL as a function of temperature measured using TGA.

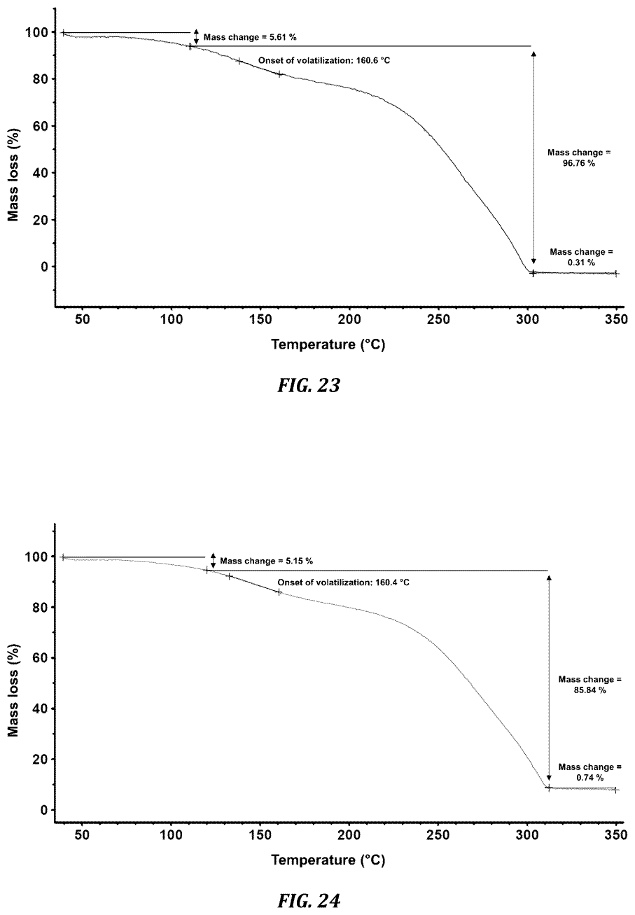

FIG. 23 shows the volatilization/degradation of the [Ni(FuranC₈-DGE)₆][Ni(hfacac)₃]₂ MIL as a function of temperature measured using TGA

FIG. 24 shows the volatilization/degradation of the [Mn(FuranC₈-DGE)₆][Mn(hfacac)₃]₂ MIL as a function of temperature measured using TGA

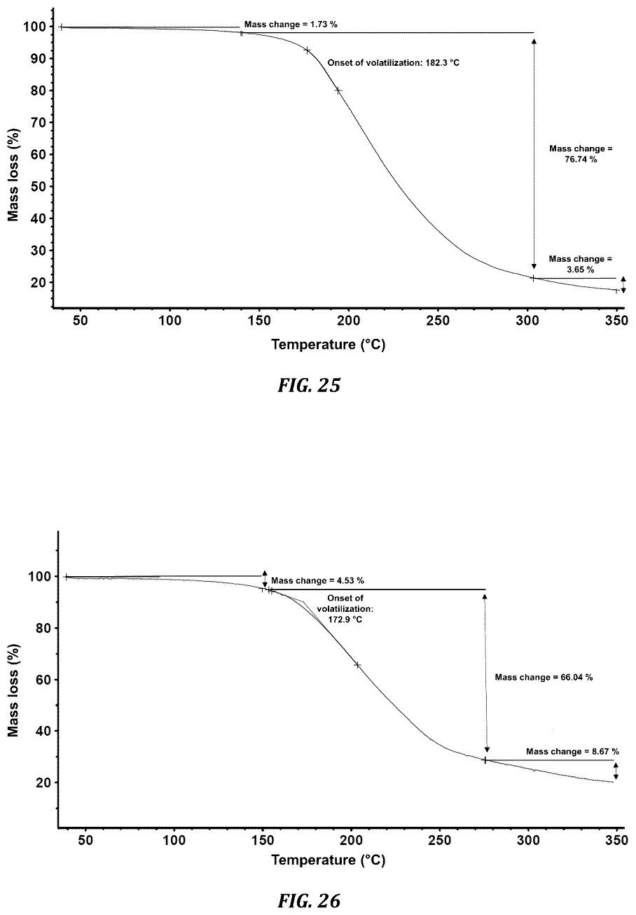

FIG. 25 shows the volatilization/degradation of the [Co(C₈-DGE)₆][Dy(hfacac)₄]₂ MIL as a function of temperature measured using TGA.

FIG. 26 shows the volatilization/degradation of the [Co(C₈-DGE)₆][Gd(hfacac)₄]₂ MIL as a function of temperature measured using TGA.

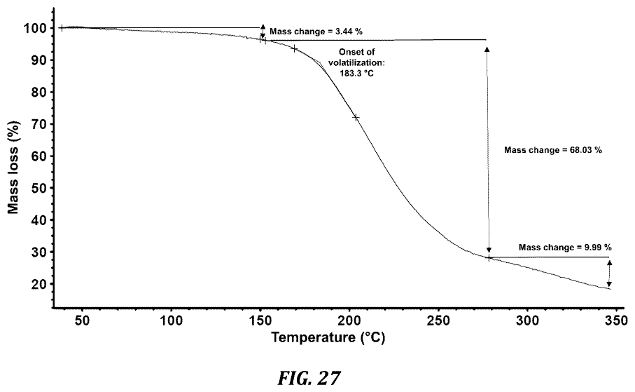

FIG. 27 shows the volatilization/degradation of the [Co(C₈-DGE)₆][Ho(hfacac)₄]₂ MIL as a function of temperature measured using TGA.

Additionally, TABLE 3 provides the onset temperature of MIL volatilization/degradation obtained from TGA measurements at 10% weight loss.

Degradation temperatures were observed in the range from 163.0 to 264.4° C. and MILs comprised of the C₈-DGE ligand offered slightly higher thermal stabilities compared to those featuring C₆-DGE. MILs comprised of CycloC₆-DGE degraded at temperatures that were approximately 50° C. lower than the volatilization temperatures of MILs containing the C₆-DGE ligand. Additionally, no significant variations in degradation temperature were observed when Co, Ni, or Mn metals were employed in the cation/anion. However, substituting transition metals with lanthanides in mixed metal MILs (MILs 13 to 15) resulted in lower volatilization temperatures.

To further investigate the thermal stability of MILs using a more sensitive technique, the solvents were employed as a gas chromatographic (GC) stationary phase and subjected to an oven temperature program from 40 to 200° C. at a ramp rate of 1° C. min⁻¹ and decomposition/degradation products detected using an ultra-sensitive flame ionization detector (FID).

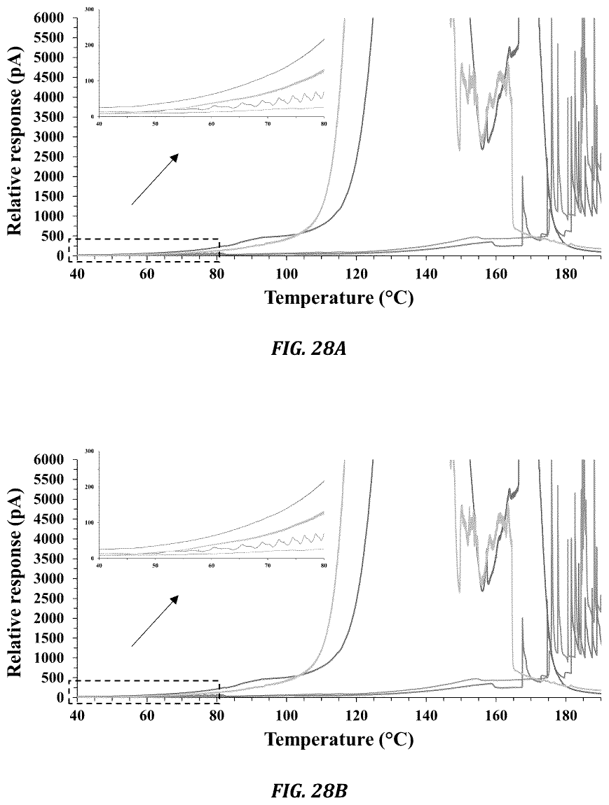

FIG. 28 shows the FID detector response as a function of temperature for select MILs. From FIG. 28A, it was observed that the Co MIL comprised of the CycloC₆-DGE ligand exhibited higher detector response due to decomposition products entering the detector at lower temperatures compared to the same MIL comprised of C₆-DGE ligand, indicating stronger metal-ligand interactions offered by the straight-chained chelating groups. However, no significant difference in detector response was observed for MILs 4 to 6, which varied in the type of transition metal employed in the cation/anion, as shown in FIG. 28B.

The thermal stability diagram for DGE-based MILs obtained by recording the detector response of a flame ionization detector as a function of temperature when the MIL is used as a gas chromatographic stationary phase shown in FIGS. 28A-28B is a red line for [Co(C₆-DGE₆)][Co(hfacac)₃]₂; a blue line for [Co(CycloC₆-DGE₆)][Co(hfacac)₃]₂; an orange line for [Co(C₈-DGE₆)][Co(hfacac)₃]₂; a teal line for [Mn(C₈-DGE₆)][Mn(hfacac)₃]₂; and a yellow line for [Co(C₈-DGE₆)][Ho(hfacac)₄]₂. FIG. 28A shows the effect of cationic ligands on MIL degradation and decomposition and FIG. 28B provides a comparison of transition metal MILs with mixed metal combinations.

Additionally, the [Co(C₈-DGE)₆][Gd(hfacac)₄]₂ and [Co(C₈-DGE)₆][Ho(hfacac)₄]₂ MILs exhibited very similar responses and volatilized at lower temperatures of approximately 120° C. TABLE 3 summarizes the onset temperature of MIL volatilization/degradation obtained using GC-FID measurements. It was observed that the values obtained from these experiments were approximately 30-50° C. lower compared to those measured using TGA. Nevertheless, the trends observed using both techniques were in agreement in most of the cases.

From the foregoing, it can be seen that the present disclosure accomplishes at least all of the stated objectives.

The inventions being thus described, it will be obvious that the same may be varied in many ways. Such variations are not to be regarded as a departure from the spirit and scope of the inventions and all such modifications are intended to be included within the scope of the following claims.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

What is claimed is:

1. A magnetic ionic liquid (MIL) comprising:
a cationic ligand comprising a diglycolic acid ester having the following formula (I) 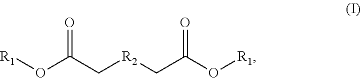
wherein R₁ comprises an organic substituent having from about 2 carbons to about 24 carbons; and wherein R₂ is an N—CH₃, O, S, or thiophene; wherein the cationic ligand chelates a metal, and wherein the metal comprises nickel (Ni), copper (Cu), cobalt (Co), iron (Fe), manganese (Mn), chromium (Cr), vanadium (V), dysprosium (Dy), holmium (Ho), gadolinium (Gd), neodymium (Nd), europium (Eu), cerium (Ce), thulium (Tm), ruthenium (Re), terbium (Tb), erbium (Er), ytterbium (Yb), and a salt or a mixture thereof;
an anionic ligand having the following general formula (II)
[M(Y)ₓ⁻] (II)
wherein M is transition metal or rare earth metal ion; and Y is a chelating agent having the general formula (III): 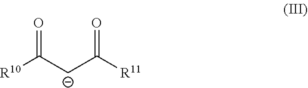
each of the R¹⁰ and R¹¹ are independently a substituted or unsubstituted methyl, phenyl, thiophenyl, napthyl, alkyl, or aryl group; and x is 3 or 4;
wherein M comprises nickel (Ni), copper (Cu), cobalt (Co), iron (Fe), manganese (Mn), chromium (Cr), vanadium (V), dysprosium (Dy), holmium (Ho), gadolinium (Gd), neodymium (Nd), europium (Eu), cerium (Ce), thulium (Tm), ruthenium (Re), terbium (Tb), erbium (Er), ytterbium (Yb), and salts or mixtures thereof.

2. The MIL of claim 1, wherein the anionic ligand comprises one or more of the following: 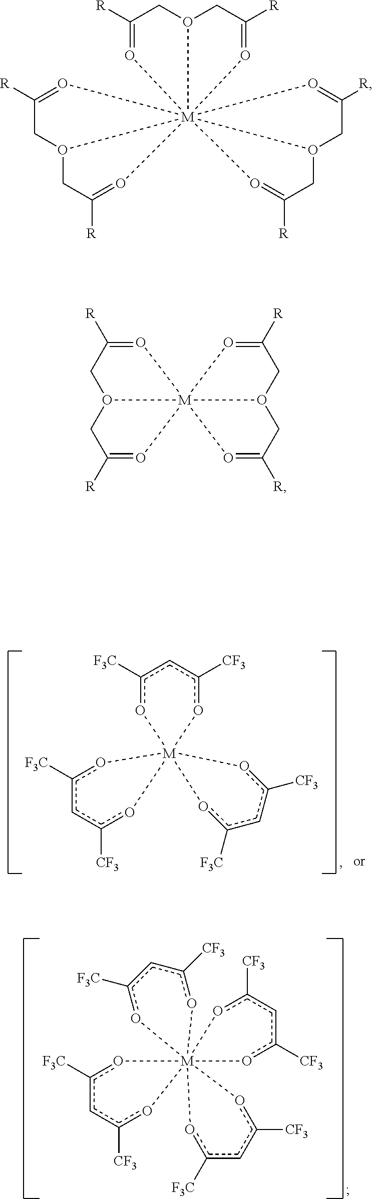
wherein R is one or more of the following: 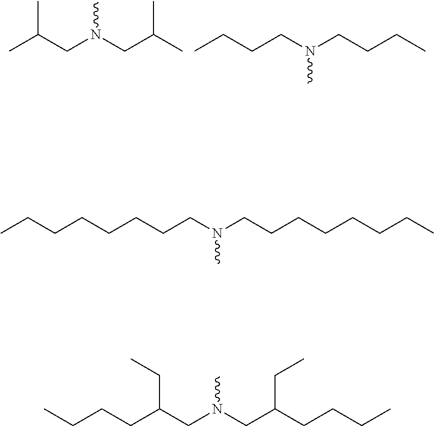
and wherein M comprises nickel (Ni), copper (Cu), cobalt (Co), iron (Fe), manganese (Mn), chromium (Cr), vanadium (V), dysprosium (Dy), holmium (Ho), gadolinium (Gd), neodymium (Nd), europium (Eu), cerium (Ce), thulium (Tm), ruthenium (Re), terbium (Tb), erbium (Er), ytterbium (Yb), and a salt or a mixture thereof.

3. The MIL of claim 2, wherein the cationic ligand comprises one or more of C₆-DGE, C₈-DGE, cyclo-C₆-DGE, and furan-C₈-DGE.

4. The MIL of claim 2, wherein the anionic ligand is 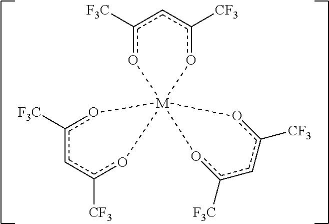
and M is Co, Ni, Mn, or a mixture thereof.

5. The MIL of claim 2, wherein the anionic ligand is 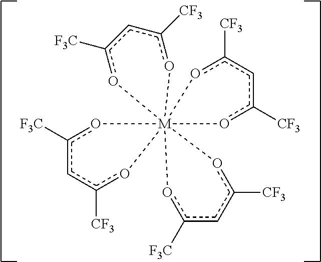
and M is Dy, Gd, Ho, or a mixture thereof.

6. The MIL of claim 1, wherein the MIL has a magnetic susceptibility of at least about 1000 10⁻⁶ cm³ mol⁻¹.

7. The MIL of claim 1, wherein the MIL has a viscosity of less than about 150 cp at the temperature of about 23.6° C.

8. A kit for detecting, concentrating and/or extracting microbes in a sample comprising:
the magnetic ionic liquid of claim 1;
an extracting medium; wherein the extracting medium is a Luria-Bertani-derived nutrient broth comprising more than 10 g/L of tryptone, 10 g/L of NaCl, 5 g/L of yeast extract, or combination thereof; wherein a volume ratio between the magnetic ionic liquid and the extracting medium is from about 1:5 to 1:15; and
a power-free heat source.

9. The kit according to 8, wherein the kit further comprises an enzyme for Recombinase Polymerase Amplification (RPA).

10. The kit according to claim 8, wherein the power-free heat source is a chemical heat source, a battery-power heat source, a solar-powered heat source, or combination thereof.

11. A method of detecting, concentrating and/or extracting microbes comprising:
contacting a sample with a magnetic ionic liquid (MIL) for the period of a contacting time; wherein the MIL is the MIL of claim 1;
extracting the microbes or their diagnostically useful cellular components from the MIL using an aqueous extracting medium to generate an extracted microbe sample;
detecting the microbes; wherein the detecting is performed by an amplification-based detection method, a non-amplification-based detection method, or both;
wherein the sample comprises viable microbes and wherein the viable microbes comprise gram-negative bacteria, gram-positive bacteria, or a mixture thereof.

12. The method according to claim 11, wherein the detecting is performed by an amplification-based detection method.

13. The method according to claim 11, wherein the microbes are gram-negative bacteria selected from the group consisting of Cronobacter sakazakii, E. coli, Klebsiella aerogenes, Pantoea eucalypti, Pantoea stewartii, Pectobacterium carotovorum, Salmonella bongori, Salmonella enterica, Serratia marcescens, Yersinia enterocolitica, and mixtures thereof.

14. The method of claim 11, wherein the sample is an aqueous solution derived from food, water, or aerosol as a result of dilution, extraction, soaking, rinsing, washing, collecting, concentrating, or combination thereof; and wherein the magnetic ionic liquid extracts the viable microbes from the sample.

15. The method of claim 14, wherein the extracting medium is a Luria-Bertani-derived nutrient broth comprising more than 10 g/L of tryptone, more than 5 g/L of yeast extract, more than 10 g/L of NaCl, or combination thereof.

16. The method of claim 15, wherein a volume ratio between the magnetic ionic liquid and the extracting medium is from about 1:5 to 1:15.

17. The method of claim 16, wherein the amplification-based detection method comprises using Recombinase Polymerase Amplification (RPA) on the extracted microbe sample for amplifying DNA or RNA of the microbes; wherein the RPA is carried out with a power-free heat source; and wherein RPA is applied to the extracted microbe sample for amplifying characteristic DNA of the microbes at a temperature of from about 20° C. to about 50° C.

18. The method of claim 17, wherein the microbes are Salmonella and the RPA comprises using a DLH primer.

19. The method of claim 17, wherein the RPA comprises using a reverse transcriptase (RT) enzyme for characteristic RNA of the microbes.

20. The method of claim 11, wherein the detection is done through a nucleic acid lateral flow immunoassay (NALFIA) or gel electrophoresis.
